(19) United States
(12) Patent Application Publication
(10) Pub. No.: US 2025/0009617 A1
(43) Pub. Date: Jan. 9, 2025

(54) Oral Care Compositions With Anticaries Activity

(71) Applicant: The Procter & Gamble Company, Cincinnati, OH (US)

(72) Inventors: Samuel James St. John, Cincinnati, OH (US); Arif Ali Baig, Mason, OH (US); Aaron Reed Biesbrock, Maineville, OH (US)

(21) Appl. No.: 18/886,278

(22) Filed: Sep. 16, 2024

Related U.S. Application Data
(63) Continuation of application No. 17/037,780, filed on Sep. 30, 2020.
(60) Provisional application No. 62/994,893, filed on Mar. 26, 2020.
(60) Provisional application No. 62/985,451, filed on Mar. 5, 2020.
(60) Provisional application No. 62/972,109, filed on Feb. 10, 2020.
(60) Provisional application No. 62/972,111, filed on Feb. 10, 2020.
(60) Provisional application No. 62/943,940, filed on Dec. 5, 2019.
(60) Provisional application No. 62/907,733, filed on Sep. 30, 2019.
(60) Provisional application No. 62/907,735, filed on Sep. 30, 2019.
(60) Provisional application No. 62/907,736, filed on Sep. 30, 2019.

(51) Int. Cl. A61K 8/27; A61K 8/20; A61K 8/21; A61K 8/24; A61K 8/35; A61K 8/362; A61K 8/365; A61K 8/44; A61K 8/73; A61K 8/81; A61K 8/97; A61K 8/9789; A61Q 11/00

(52) U.S. Cl. CPC A61K 8/27; A61K 8/20; A61K 8/21; A61K 8/24; A61K 8/35; A61K 8/362; A61K 8/365; A61K 8/44; A61K 8/731; A61K 8/8147; A61K 8/97; A61K 8/9789; A61Q 11/00; A61K 2800/28; A61K 2800/30; A61K 2800/48; A61K 2800/74; A61K 2800/92

(57) ABSTRACT

Oral care compositions with two or more components with a subtherapeutic anticaries effect, but, in combination, provide a therapeutic anticaries effect. Oral care compositions that are fluoride-free or lower in fluoride content, but have an equivalent or greater anticaries benefit than a composition comprising a therapeutic amount of fluoride ions.

### FIELD OF THE INVENTION

The present invention is directed to compositions with anticaries activity. The present invention is also directed to compositions with two or more anticaries agents that are individually subtherapeutic, but that have an overall therapeutic anticaries activity. The present invention is also directed to fluoride-free compositions with an equivalent or greater anticaries benefit than a composition comprising a therapeutic amount of fluoride ions. The present invention is also directed to compositions comprising a therapeutic amount of fluoride and another anticaries agent with a greater anticaries benefit than a composition comprising a therapeutic amount of fluoride ions as the sole anticaries agent.

### BACKGROUND OF THE INVENTION

The current consumer goods marketplace reflects an increasing awareness of the entire lifecycle of a product, including the provenance of the various ingredients, packaging, and research methods used to substantiate a product's effectiveness. Consumers are rejecting petrochemically derived ingredients and shifting the marketplace to responsible-sourced and naturally-derived raw materials, recyclable packaging, and minimally processed materials. Coupled with this trend is a rejection of fluoride for concerns, real or imagined, of its toxicity in drinking water, toothpaste, or both. This trend has provably led to increases in cavities in consumers whom reject fluoride because there are no viable alternatives to fluoride in over-the-counter oral care products for the prevention of cavities. Consequently, the current marketplace requires that consumers trade clean (giving up fluoride) for effective (anticavity toothpaste).

As such, there is a need in the art for a fluoride-free, anticavity toothpaste for consumers who reject fluoride in drinking water and/or toothpaste. Additionally, there is a need in the art for the enhancement for fluoride's effectiveness in a fluoride-containing toothpaste for consumers that reject fluoride in the drinking water, but still use a therapeutically effective level of fluoride in an anticavity toothpaste. There is also a need in the art for the enhancement of fluoride's activity in a situation where fluoride is formulated at a subtherapeutic level in order to reduce overall fluoride exposure, which is desirable situation for young children using anticavity toothpaste. There is also a need in the art to deliver the above compositions using naturally derived ingredients whose efficacy is traditionally significantly deficient that of traditional therapeutic levels of fluoride (i.e., 1100 ppm F as NaF in a silica-based toothpaste).

### SUMMARY OF THE INVENTION

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

Disclosed herein is an anticavity, fluoride-free oral care composition, wherein the composition has a rat caries score of about 60% or more of a rat caries score of a positive control oral care composition, the positive oral care composition comprising 1100 ppm of sodium fluoride.

Also disclosed herein is an oral care composition comprising (a) a first subtherapeutic anticaries agent (b) a second subtherapeutic anticaries agent, wherein the oral care composition is free of a fluoride source and the first and second subtherapeutic anticavity agents collectively have a therapeutic anticavity benefit.

Also disclosed herein is an oral care composition comprising (a) an anticaries drug composition comprising fluoride; and (b) a subtherapeutic anticaries agent, wherein the subtherapeutic anticaries agent is free of fluoride, wherein the oral care composition has a therapeutic benefit greater than the anticaries drug composition.

### BRIEF DESCRIPTION OF THE DRAWINGS

FIG. 1 shows rat caries results for the invention relative to the fluoride control products.

FIG. 2 shows iPGRM dose response of chlorhexidine (% acid reduction v Crest Cavity Protection) along with the iPGRM performance of the novel compositions

FIG. 3 shows iPUM behavior of different calcium sources (ratio of biofilm calcium content following treatment relative to the biofilm calcium content following treatment with Crest Cavity Protection) along with the iPUM performance of the novel compositions

FIG. 4 shows F-Free HAP solubility reduction with respect to Sn content along with the F-Free HAP Solubility reduction of typical Sn/silica toothpaste compositions.

FIG. 5 shows actual v predicted rat caries percent reduction vs. placebo (left) and the plot of residuals (right).

### DETAILED DESCRIPTION OF THE INVENTION

Caries is a process by which tooth damage occurs by exposure to plaque acids and the subsequent net demineralization of the tooth. Bacteria in the plaque produce organic acids from the metabolism of fermentable carbohydrates to which they have access when a person cats. Consequently, avenues for intervention in caries generally involve the suppression of acid formation and/or the stabilization of the tooth surface. In the present invention, we have considered four mechanisms through which this may occur, namely: i) suppressing acid formation via antibacterial action; ii) reducing enamel solubility through a calcium co-ion effect; iii) reducing enamel solubility through a fluoride co-ion effect; and iv) reducing enamel solubility through surface adsorbed stabilizers. These mechanisms are considered essential for the reduction in human cavities. Until now, the one favored by nearly every anti-cavity product on the market is exclusively the reduction of enamel solubility through a fluoride co-ion effect.

Thus, the present invention is directed at compositions that can treat caries and prevent cavities comprising two or more subtherapeutic anticaries agents, such as an antibacterial agent, a surface-adsorbed enamel stabilizing agent, a calcium ion source, and/or a fluoride ion source. Additionally, the present invention is directed at compositions with improved anticaries activity comprising a therapeutic amount of fluoride ions and one or more subtherapeutic anticaries agents, such as an antibacterial agent, a surface-adsorbed enamel stabilizing agent, or calcium ion source.

Additionally, the present invention is directed to creating a therapeutic oral care composition that is a combination of two or more sub-therapeutic compositions using a model for the performance of oral care compositions generally in rat caries along the vectors of fluoride co-ion effect, calcium co-ion effect, Sn-free antibacterial efficacy, and F-Free hydroxyapatite solubility reduction via surface adsorption. Such a model will allow the rapid, ethical, and responsible identification of ingredients that minimizes or virtually eliminates animal testing for new therapeutic anti-caries oral care compositions to create fluoride-free and enhanced-fluoride compositions.

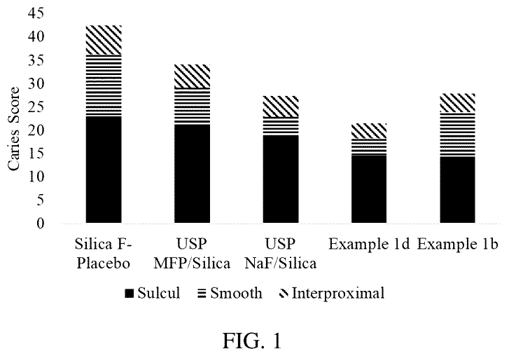

Since the discovery of fluoride there have been numerous attempts in the art to find alternatives to, or enhancements for, fluoride. Those attempts have generally favored one of the four mechanisms listed above, most especially plaque acid suppression. The disclosed methods and compositions of the present application are radically different from what was presented in the art. While not wishing to be bound by theory, it is believed that subtherapeutic amounts of compositions supplied simultaneously would deliver, in total, a composition capable of reducing cavities. Thus, the present invention is directed to compositions comprising at least two subtherapeutic compositions in accord with the mechanisms described above, yet the disclosed compositions delivered a reduction in caries as measured in the rat caries model at least equivalent to 1100 ppm F as sodium fluoride and superior to that of the 1100 ppm F control when fluoride was included, as shown in FIG. 1. Additionally, the disclosed compositions led to a reduction of caries in the rat sulcus and interproximal spaces that exceeded the effectiveness of fluoride. This unexpected result demonstrated that this approach was novel as nowhere in the art was it taught that a combination of subtherapeutic compositions could be effective for the prevention of cavities.

The rat caries results, presented in FIG. 1, indicated that Ex 1b reduced caries relative to sodium fluoride/silica and monofluorophosphate/silica compositions in a placebo-controlled study. Ex. 1b which did not have fluoride, had a total caries reduction equivalent to the sodium fluoride/silica control product; however, it had an unexpectedly large reduction in sulcal caries relative to control.

The disclosed compositions were designed to work on the tooth and in the biofilm with a collection of subtherapeutic compositions. This has led to a remarkable reduction in sulcal and interproximal caries with respect to the control compositions. When fluoride is added to the disclosed compositions, as in Ex. 1d, the reduction in caries is enhanced with a notable reduction in smooth surface caries.

It was unexpectedly found that the non-fluoride mechanisms can have a large contribution to the reduction in caries considering that such an approach had not be disclosed in the art. In order to better understand this result, a set of laboratory tests were performed to measure the contribution of each subtherapeutic composition to the composition tested in the rat. Individual methods have been developed to measure the efficacy of compositions along the above described intervention vectors. These methods are the: i) Sn-Free in vitro plaque glycolysis and regrowth method (Sn-Free iPGRM); ii) in vitro plaque uptake method for calcium (iPUM-Ca); iii) F-Free hydroxyapatite solubility reduction method (F-Free HAP); and iv) ADA one-minute fluoride release (ADA). A person of ordinary skill in the art would recognize that some ingredients, such as Sn, have both an antibacterial effect and a HAP surface stabilization effect. Such behavior complicates the analysis of a composition's performance; therefore, the entire contribution of such ingredients is considered through a single mechanism only. Thus, for the purposes of this invention, a composition's antibacterial efficacy should be determined with respect to its Sn placebo or by other experimental design approaches that correctly account single variably for the contribution of Sn in the iPGRM. Similarly, for the purposes of this invention, a composition's ability to reduce hydroxyapatite solubility should be determined using the fluoride-free version of the composition (if it contains fluoride) or by some other experimental design that correctly controls for fluoride's contribution. TABLE 2 illustrated the results of the characterization of the novel compositions illustrated in TABLE 1 and control toothpastes using the four different methods indicated above with their corresponding rat caries scores, as disclosed in the Example section.

During the discovery of fluoride, several rat caries tests were run exploring both the effectiveness of fluoride as well as alternatives. The catalogue of rat caries experiments were meta-analyzed to illustrate the single variable behavior of the previously mentioned intervention vectors. As will be illustrated later, this let us evaluate the efficacy of different vectors relative to a therapeutic level of fluoride.

TABLE 3 shows the variation in anticaries efficacy with respect to soluble fluoride content as determined by the ADA method. Using this example and the meta-analysis, a 650 ppm fluoride as sodium fluoride was estimated to effect a reduction in caries of about 29%, or about 25%, or about 30%, with respect to the placebo or water control in rat caries experiments.

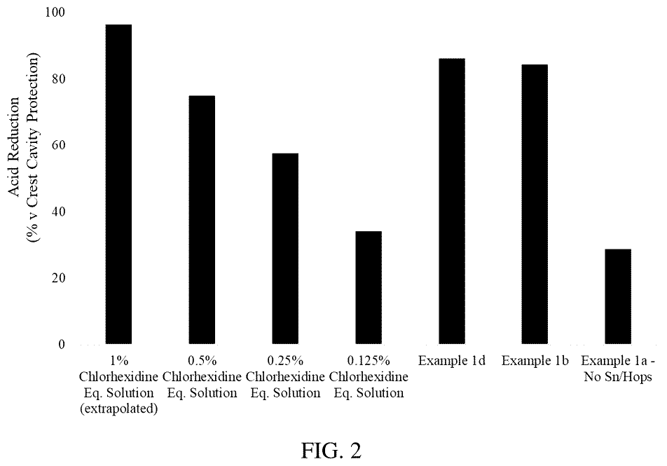

TABLE 4 shows the variation in anticaries efficacy with respect to Sn-Free antibacterial activity as measured by the Sn-Free iPGRM using the dose responsive reduction in rat caries from a well-known antibacterial, chlorhexidine. These compositions were then analyzed using iPGRM to define the variation in rat caries effectiveness relative to its antibacterial efficacy. Since the iPGRM measures reduction in antibacterial efficacy through changes in the pH, non-antibacterial agents that buffer pH, such as sodium bicarbonate, can be corrected for when determining the efficacy of the antibacterial agent. This can be done using placebo controls as appropriate to isolate the Sn-Free antibacterial contribution. The contribution of Sn was measured through the solubility reduction and is excluded from this test. The iPGRM dose response of chlorhexidine is shown in FIG. 2.

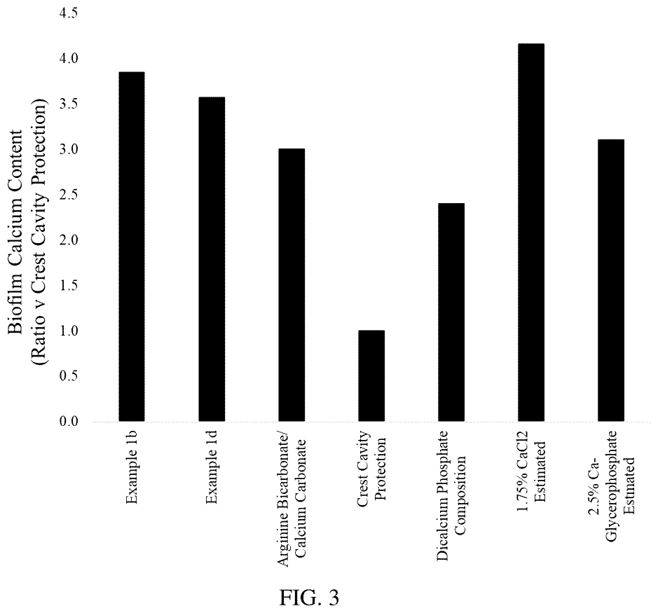

TABLE 5 shows the variation in anticaries efficacy with respect to calcium co-ion effect as measured by the iPUM. This method measures the total calcium uptake of both soluble and insoluble sources. If an insoluble source was used, calcium sources were preferred that were substantially more soluble than hydroxyapatite so that they will dissolve preferentially when exposed to plaque acids. For example, dicalcium phosphate dihydrate or calcium carbonate dissolve readily on exposure to acid while calcium pyrophosphate is a poor source of calcium for the purposes here. Insoluble sources have the advantage of increased residence time in the plaque and can, therefore, provide a bloom of calcium to correspond temporally to the generation of plaque acid. The iPUM dose response of calcium-containing compositions is shown in FIG. 3.

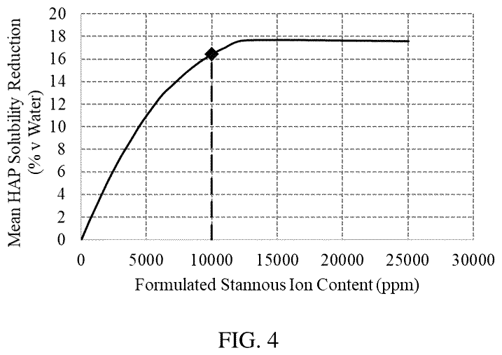

TABLE 6 shows the variation in anticaries efficacy with respect to the F-Free reduction in HAP solubility as measured by F-free HAP. The contribution of the fluoride co-ion effect is measured through the ADA method and is excluded from this test. An example of the F-free HAP response for different levels of Sn in Sn/silica toothpastes is given in FIG. 4.

Finally, the threshold can be defined for which combinations of subtherapeutic compositions result in a therapeutic composition. On average, therapeutic compositions can be defined as those providing a reduction in caries at least about equivalent to that of a 650 ppm available F as NaF/silica toothpaste and/or at least about 24%, 25%, 26%, 27%, 28%, 29%, 30%, 31%, 32%, 33%, or 34% reduction in caries. Caries reduction and/or anticavity activity can be substantiated using an actual rat caries performance test. This threshold can be a guide during development of new compositions for when reductions achieve an approximately similar therapeutic level of benefit to that of a 650 ppm available F as NaF/silica toothpaste with respect to the placebo or water control.

With these methods, the efficacy can be quantified of various compositions along the important anticavity intervention methods. The same analysis can be retrospectively applied to a long history of rat caries experiments conducted during the original development of fluoride-containing anticavity compositions. Approximately 170 rat caries experiments were analyzed containing over 800 individual treatments for the reductions of caries in two different, but similar, rat caries models. These experiments were conducted between 1959 and 2019. Rat caries is the preferred animal model for human caries and is included in the US Anticaries Monograph (21 CFR part 355) to ensure the efficacy of fluoride-containing products. It is additionally sensitive to non-fluoride anticaries mechanisms and compositions. Finally, animal models for caries are generally considered interchangeable as long as they have been properly developed.

This retrospective analysis can help define the scope of the present invention. As described herein, the present invention is directed to a combination of subtherapeutic compositions that can result in a therapeutic composition when tested in total. The examples can allow for the setting of performance thresholds for the subtherapeutic to therapeutic transition in each mechanism as defined by the specified performance test. However, those results, alone, might not anticipate the combined performance of the compositions in a rat caries experiment. The analysis gave a model from which can predict reductions in rat caries for combinations of subtherapeutic compositions as described herein. In combination, the examples, performance thresholds, and mathematical model can help define the present invention.

The retrospective analysis of rat caries experiments was possible because of detailed descriptions of the treatments/ingredients with additional measurements of F content, Sn content, and pH frequently documented in the experimental record. Efficacy measures in the various anticaries mechanism methods described above were either measured directly when the ingredients could be obtained or estimated using the detailed description of the treatments and comparisons to similar present-day compositions. Estimations of rat caries efficacy using the four methods named above resulted in a model correlation coefficient, r², of ˜0.76. The correlation coefficient suggests that 76% of the variation in rat caries efficacy is captured by these methods. The remaining 24% of variation is typically ascribed to variation commonly observed in biological methods. Suffice it to say, we believe this represents a good model for the performance of various compositions in rat caries. The prediction formula is given below for the % reduction in rat caries with respect to water or a silica-based-abrasive, placebo-toothpaste negative control.

$$\% Reduction = 1.77 * sqrt ( ADA / 2 ) + 0.146 * iPGRM + 3.97 * iPUM - Ca + 0.689 * HAP - 6.84 \qquad Formula 1$$

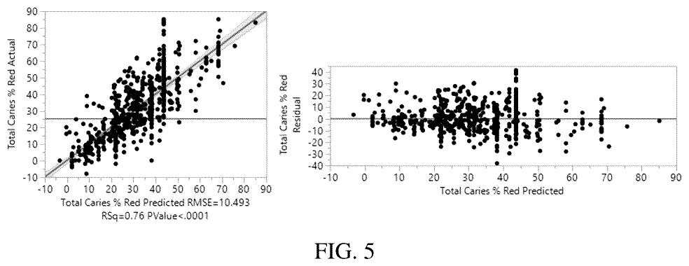

The predicted versus actual values for the retrospective analysis of rat caries data are given in FIG. 5a with a plot of the residuals in FIG. 5b. When a sodium monofluorophosphate (MFP) toothpaste is used, 16 is subtracted from the % Reduction calculated using Formula. This is done because MFP is less effective in the rat caries model.

This approach is novel and not obvious to those in the art for many reasons:

Firstly, there is a long-standing need for a fluoride-free anticavity toothpaste as evidenced by the distinct lack of such a product in the market. There are no anti-cavity products in the United States legally marketed without a therapeutic level of fluoride. This means that no one has successfully completed a new drug application that meets the rigorous FDA standards of safety and efficacy. And as such, there has been a long-felt need for such an alternative.

Secondly, the application methods and model described herein are not enough alone to make a therapeutic composition. Some combinations of subtherapeutic compositions are not straightforwardly compatible with one another. For example, soluble fluoride sources are frequently deactivated by calcium unless special care is taken. Cationic antibacterial agents can be deactivated by anionic surfactants. The use of stannous ion sources can necessitate the careful choice of stabilizers and packaging to prevent loss and/or oxidation. Calcium pyrophosphate does not readily dissolve on exposure to plaque acid and is a poor source of calcium for the purposes of protecting teeth from plaque acids through a co-ion effect. There are many more examples like this that require expertise in the art to overcome.

Thirdly, nearly all previous examples of anti-cavity compositions in the art focused on how much of a single composition was required to provide a therapeutic benefit. None of the examples in the art teach how little you can use to achieve a therapeutic benefit from combinations of subtherapeutic compositions. This is important in cases where exposure of some ingredients should be limited because of toxicity, aesthetic, or cost concerns. One example comes close by showing a combination of a solubility reducing composition and an antibacterial composition (US 2018/0028417 A1); however, it did not teach about the combination of subtherapeutic compositions to achieve a therapeutic effect. Additionally, the solubility reducing composition was included at an already therapeutic level to which a second composition enhanced its effect.

Fourthly, no single example taught a model, method, strategy, or approach to achieve therapeutic levels of activity through combinations of subtherapeutic compositions. Furthermore, in none of these art examples, alone or together, are a method, model, or strategy to achieve therapeutic levels of activity through combinations of subtherapeutic compositions taught. In some of the disclosed patents, common subtherapeutic agents are disclosed; however, never to a degree such that a systematic analysis can reveal their impact and interaction leading to a rational design of subtherapeutic compositions. The performance of subtherapeutic compositions, excipients, and combinations thereof are frequently diminished, controlled for experimentally, or just plainly left uninvestigated. In no case are they explicitly optimized for and to the best of our knowledge this has not been done in a systematic way. It is exceedingly unlikely, considering the limited history of rat caries studies published in the art, such an analysis could be performed by a person of skill in the art by merely evaluating existing studies in the published art.

The possible anticaries agents are further described herein.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “therapeutic anticaries activity”, as used herein, is the anticaries activity provided by a composition including an anticaries drug and/or one or more anticaries agents in a therapeutic dose. A therapeutic dose for fluoride ions is defined in the United States by the Food and Drug Administration (FDA) Monograph (21 CFR part 355). For example, a therapeutic amount of sodium fluoride in a paste dosage form (i.e. paste dentifrice) is 850 to 1,150 ppm with an available fluoride ion concentration of at least 650 ppm. Other anticaries drugs are disclosed in 21 CFR part 355, which is herein incorporated by reference. Other therapeutic dosages are available in respective jurisdictions. Thus, as used herein, therapeutic anticaries activity for a composition comprising one or more anticaries agents is the anticaries benefit which is equivalent or better than the anticaries benefit provided by a composition comprising sodium fluoride with at least 650 ppm of available fluoride ions.

The term “subtherapeutic anticaries activity”, as used herein, is the anticaries activity of a composition that has a lower anticaries benefit relative to the anticaries benefit provided by a composition comprising sodium fluoride with at least 650 ppm of available fluoride ions, as defined in “therapeutic anticaries drug composition.” The term “subtherapeutic anticaries activity” also can also describe anticaries agents that can, in combination with additional anticaries agents, lead to a therapeutic benefit relative to the anticaries benefit provided by a composition comprising sodium fluoride with at least 650 ppm of available fluoride ions, when utilized in an oral care composition suitable for use in the oral cavity of a human, but alone has not been shown to provide a therapeutic benefit at concentrations suitable for use in an oral care composition.

The term “anticaries drug”, as used herein, is a drug that aids in the prevention and prophylactic treatment of dental cavities (decay, caries). This can include fluoride ion sources, such as in 21 CFR part 355 and anticaries agents, as described herein.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63 (5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The dentifrice composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

Section headers are provided below for organization and convenience only. The section headers do not suggest that a compound cannot be within more than one section. In fact, compounds can fall within more than one section. For example, stannous chloride can be both a tin ion source and a surface adsorbed stabilizer, stannous fluoride can be both a tin ion source and a fluoride ion source, glycine can be an amino acid, a buffering agent, and/or a surface adsorbed stabilizer, among numerous other compounds that can fit amongst several categories and/or sections.

### Anticaries Activity

The oral care compositions, as described herein, can comprise one or more anticaries agents, which collectively demonstrate therapeutic anticaries activity. As described herein, therapeutic anticaries activity is defined by the relevant regulatory agency in a jurisdiction of interest, such as the U.S. FDA in the United States. At the FDA, a therapeutic amount of sodium fluoride in a paste dosage form (i.e. paste dentifrice) is 850 to 1,150 ppm with an available fluoride ion concentration of at least 650 ppm. Thus, the oral care compositions of the present invention can have an anticaries benefit at least about of the anticaries benefit of a composition comprising at least, at least about, or greater than 650 ppm, 800 ppm, 850 ppm, 1100 ppm, 1150 ppm, 1450 ppm, and/or 2800 ppm of free fluoride ions, which can correspond to therapeutic anticaries activity.

The anticaries activity of the oral care compositions, as described herein, can also be described by a rat caries score. The oral care compositions can have a rat caries score of about 35 or less, about 30 or less, about 25 or less, and/or about 20 or less. The anticaries activity of the oral care compositions, as described herein, can also be described the percent reduction of the rat caries score relative to a placebo toothpaste. The oral care compositions can have a percent reduction in caries relative to a placebo toothpaste (i.e. a negative control) of at least about 25%, at least about 29%, at least about 30%, at least about 35%, and/or at least about 40%.

The anticaries activity of the oral care compositions, as described herein, can also be described by a percent reduction of the rat caries score relative to an oral care composition comprising a therapeutic dose of an anticaries agent (i.e. a positive control, such as USP NaF). The oral care compositions can have a percent reduction in caries relative to a positive control, such as USP NaF, of about 50% or more, about 60% or more, about 65% or more, about 70% or more, about 75% or more, about 80% or more, about 90% or more, about 100% or more, about 125% or more, and/or about 150% or more.

### Anticaries Agent

The oral care composition, as described herein, comprise one or more anticaries compositions, the one or more anticaries compositions comprising one or more anticaries agents, which can, individually, be in a therapeutic dose or a subtherapeutic dose.

The oral care composition can comprise a first subtherapeutic anticaries agent and a second subtherapeutic anticaries agent, which in total lead to a therapeutic anticaries benefit. The first and second subtherapeutic anti caries agents can be fluoride-free, as described herein, or one of the subtherapeutic agents can comprise a subtherapeutic amount of fluoride ions.

The oral care composition can comprise a therapeutic anticaries agent comprising a fluoride ion source and a subtherapeutic anticaries agent, which is free from a fluoride ion source, where the overall oral care composition has a higher anticaries benefit than the anticaries benefit associated with the a therapeutic anticaries composition alone. The anticaries activity of a composition comprising a therapeutic anticaries agent and a subtherapeutic anticaries agent can allow for anticaries activity that is normally available with a prescription strength concentration of fluoride ions.

The anticaries agent can be active against caries through one of these four mechanisms: i) suppressing acid formation via antibacterial action; ii) reducing enamel solubility through a calcium co-ion effect; iii) reducing enamel solubility through a fluoride co-ion effect; and iv) reducing enamel solubility through surface adsorbed stabilizers. Thus, the anticaries agent can be an antibacterial agent, a calcium ion source, a fluoride ion source, a surface adsorbed stabilizer. However, a compound can fall within more than one of these categories, such as, for example, stannous chloride, which can be an antibacterial agent and a surface adsorbed stabilizer or stannous fluoride, which can be an antibacterial agent, a fluoride ion source, and a surface adsorbed stabilizer.

### Antibacterial Agent

The oral care composition can comprise one or more anticaries agents, the one or more anticaries agents can comprise one or more antibacterial agents. The antibacterial agent can be any agent that suppresses acid formation by the bacteria of dental caries. Suitable antibacterial agents include agents that those that can provide at least about an 80%, or about 30%, 60%, 65%, 75%, 85%, 90%, or 95%, reduction in pH with respect to Crest® Cavity Protection that thereby reduce caries at least about 9%, or about 1%, 6%, 7%, 8%, 10%, 11%, or 12%, with respect to the placebo or water control in rat caries experiments.

Suitable antibacterial agents include hops acids, such as hops alpha acids, hops beta acids, hydrogenated hops acids, and/or combinations thereof. Other suitable antibacterial agents include metal ion sources, such as tin ion sources, zinc ion sources, copper ion sources, and/or combinations thereof. Other suitable antibacterial agents include triclosan, extracts from any species within the genus Magnolia, extracts from any species within the genus Humulus. Other suitable antibacterial agents include hops acids, tin ion sources, benzyl alcohol, sodium benzoate, menthylglycyl acetate, menthyl lactate, L-menthol, o-neomenthol, chlorophyllin copper complex, phenol, oxyquinoline, and/or combinations thereof. Other suitable antibacterial agents include one or more amino acids, such as basic amino acids.

The oral care composition can comprise from about 0.01% to about 10%, from about 1% to about 5%, or from about 0.5% to about 15% of an antibacterial agent. Some, but not all, suitable antibacterial agents will be discussed separately.

### Surface Adsorbed Stabilizers

The oral care composition can comprise one or more anticaries agents, the one or more anticaries agents can comprise one or more surface adsorbed stabilizers. The surface adsorbed stabilizer can be any agent that can adsorb on an enamel surface. A suitable surface adsorbed stabilizer can be any compound that can provide at least a 17%, at least a 5%, or at least a 20%, at least a 30%, and/or at least a 15%, reduction in solubility relative to water in the F-free HAP dissolution method, which thereby reduces caries at least 5%, 6%, 7%, 8%, 9%, 10%, 11%, 12%, 13%, and/or 18% with respect to the placebo or water control in rat caries experiments.

Suitable surface adsorbed stabilizers include metal ion sources, such as tin ion sources, zinc ion sources, copper ion sources, aluminum ion sources, titanium ion sources and/or combinations thereof. Other suitable surface adsorbed stabilizers include bioactive materials, amino acids, and/or combinations thereof. Some, but not all, suitable surface adsorbed stabilizes will be discussed separately.

Humulus lupulus

The oral care compositions of the present invention can comprise at least one hops compound from Formula I and/or Formula IV. The compound from Formula I and/or Formula IV can be provided by any suitable source, such as an extract from Humulus lupulus or Hops, Humulus lupulus itself, a synthetically derived compound, and/or salts, prodrugs, or other analogs thereof. The hops extract can comprise one or more hops alpha acids, one or more hops iso-alpha acids, one or more hops beta acids, one or more hops oils, one or more flavonoids, one or more solvents, and/or water. Suitable hops alpha acids (generically shown in Formula I) can include humulone (Formula II), adhumulone, cohumulone, posthumulone, prehumulone, and/or mixtures thereof. Suitable hops iso-alpha acids can include cis-isohumulone and/or trans-isohumulone. The isomerization of humulone into trans-isohumulone can be represented by Formula III.

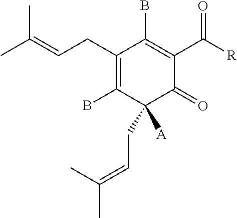

Formula I. Hops Alpha Acids. A is the acidic hydroxyl functional group in the alpha position. B arc the acidic hydroxyl functional groups in the beta position, and R is an alkyl functional group.

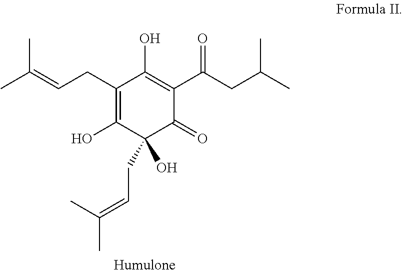 [SMILES: CC(C)=CCC1=C(O)[C@@](O)(CC=C(C)C)C(=O)C(C(=O)CC(C)C)=C1O]

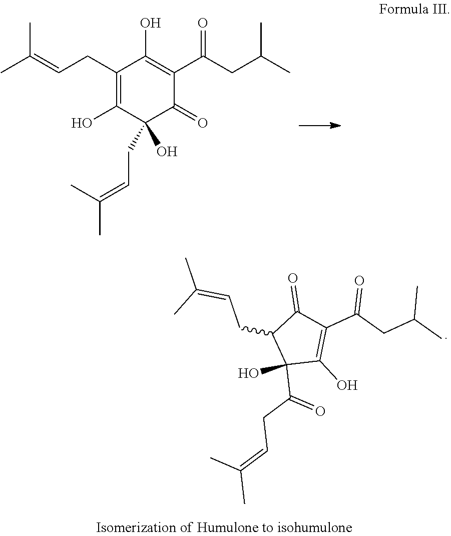

Suitable hops beta acids can include lupulone, adlupulone, colupulone, and/or mixtures thereof. A suitable hops beta acid can include a compound a described in Formula IV, V, VI, and/or VII.

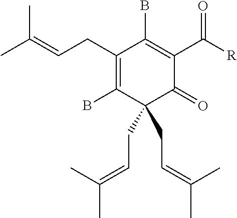

Formula IV. Hops Beta Acids. B are the acidic hydroxyl functional groups in the beta position and R is an alkyl functional group.

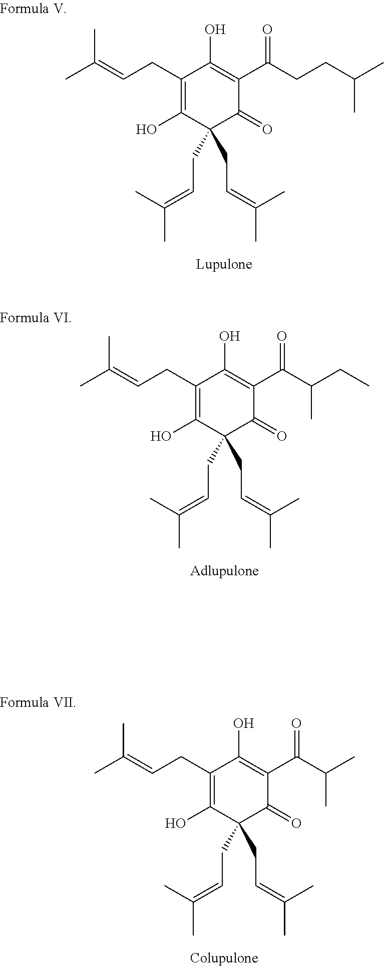

While hops alpha acids can demonstrate some antibacterial activity, hops alpha acids also have a bitter taste. The bitterness provided by hops alpha acids can be suitable for beer, but are not suitable for use in oral care compositions. In contrast, hops beta acids can be associated with a higher antibacterial and/or anticaries activity, but not as bitter a taste. Thus, a hops extract with a higher proportion of beta acids to alpha acids than normally found in nature, can be suitable for use in oral care compositions for use as an antibacterial and/or anticaries agent.

A natural hops source can comprise from about 2% to about 12%, by weight of the hops source, of hops beta acids depending on the variety of hops. Hops extracts used in other contexts, such as in the brewing of beer, can comprise from about 15% to about 35%, by weight of the extract, of hops beta acids. The hops extract desired herein can comprise at least about 35%, at least about 40%, at least about 45%, from about 35% to about 95%, from about 40% to about 90%, or from about 45% to about 99%, of hops beta acids. The hops beta acids can be in an acidic form (i.e. with attached hydrogen atom(s) to the hydroxl functional group(s)) or as a salt form.

A suitable hops extract is described in detail in U.S. Pat. No. 7,910,140, which is herein incorporated by reference in its entirety. The hops beta acids desired can be non-hydrogenated, partially hydrogenated by a non-naturally occurring chemical reaction, or hydrogenated by a non-naturally occurring chemical reaction. The hops beta acid can be essentially free of or substantially free of hydrogenated hops beta acid and/or hops acid. A non-naturally occurring chemical reaction is a chemical reaction that was conducted with the aid of chemical compound not found within Humulus lupulus, such as a chemical hydrogenation reaction conducted with high heat not normally experienced by Humulus lupulus in the wild and/or a metal catalyst.

A natural hops source can comprise from about 2% to about 12%, by weight of the hops source, of hops alpha acids. Hops extracts used in other contexts, such as in the brewing of beer, can comprise from about 15% to about 35%, by weight of the extract, of hops alpha acids. The hops extract desired herein can comprise less than about 10%, less than about 5%, less than about 1%, or less than about 0.5%, by weight of the extract, of hops alpha acids.

Hops oils can include terpene hydrocarbons, such as myrcene, humulene, caryophyllene, and/or mixtures thereof. The hops extract desired herein can comprise less than 5%, less than 2.5%, or less than 2%, by weight of the extract, of one or more hops oils.

Flavonoids present in the hops extract can include xanthohumol, 8-prenylnaringenin, isoxanthohumol, and/or mixtures thereof. The hops extract can be substantially free of, essentially free of, free of, or have less than 250 ppm, less than 150 ppm, and/or less than 100 ppm of one or more flavonoids.

As described in U.S. Pat. No. 5,370,863, hops acids have been previously added to oral care compositions. However, the oral care compositions taught by U.S. Pat. No. 5,370,863 only included up to 0.01%, by weight of the oral care composition. While not wishing to be bound by theory, it is believed that U.S. Pat. No. 5,370,863 could only incorporate a low amount of hops acids because of the bitterness of hops alpha acids. A hops extract with a low level of hops alpha acids would not have this concern.

The hops compound can be combined with or free from an extract from another plant, such as a species from genus Magnolia. The hops compounds can be combined with or free from triclosan.

The oral care composition can comprise from about 0.01% to about 10%, greater than 0.01% to about 10%, from about 0.05%, to about 10%, from about 0.1% to about 10%, from about 0.2% to about 10%, from about 0.2% to about 10%, from about 0.2% to about 5%, from about 0.25% to about 2%, from about 0.05% to about 2%, or from greater than 0.25% to about 2%, of hops beta acid, as described herein. The hops beta acids can be provided by a suitable hops extract, the hops plant itself, or a synthetically derived compound. The hops beta acid can be provided as neutral, acidic compounds, and/or as salts with a suitable counter ion, such as sodium, potassium, ammonia, or any other suitable counter ion.

The hops beta acid can be provided by a hops extract, such as an extract from Humulus lupulus with at least 35%, by weight of the extract, of hops beta acid and less than 1%, by weight of the hops extract, of hops alpha acid. The oral care composition can comprise 0.01% to about 10%, greater than 0.01% to about 10%, from about 0.05%, to about 10%, from about 0.1% to about 10%, from about 0.2% to about 10%, from about 0.2% to about 10%, from about 0.2% to about 5%, from about 0.25% to about 2%, from about 0.05% to about 2%, or from greater than 0.25% to about 2%, of hops extract, as described herein.

### Fluoride Ion Source

The oral care composition can comprise fluoride. Fluoride can be provided by a fluoride ion source. The fluoride ion source can comprise one or more fluoride containing compounds, such as stannous fluoride, sodium fluoride, titanium fluoride, calcium fluoride, calcium phosphate silicate fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of, substantially free of, or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or free of a fluoride ion source.

A subtherapeutic amount of a fluoride ion source for the purposes of anticaries activity can include an oral care composition comprising less than 1100 ppm, less than 800 ppm, less than 650 ppm, less than 500 ppm, equal to and less than 500 ppm, less than 250 ppm, equal to and less than 250 ppm, and/or combinations thereof. A subtherapeutic amount of a fluoride ion source has a measurable anticaries affect, such as at least a 5%, at least a 7.5%, at least a 10%, at least a 15%, at least a 20%, and/or combinations thereof, but not a therapeutic anticaries effect as required by a regulatory agency in any jurisdiction of interest, as described further herein.

### Tin Ion Source

The oral care composition of the present invention can comprise tin, such as from a tin ion source. The tin ion source can be any suitable compound that can provide tin ions in an oral care composition and/or deliver tin ions to the oral cavity when the dentifrice composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.5% to about 1.5%, or from about 0.3% to about 0.6%, by weight of the oral care composition, of a tin ion source.

### Ca Ion Source

The oral care composition of the present invention can comprise calcium, such as from a calcium ion source. The calcium ion source can be any suitable compound or molecule that can provide calcium ions in an oral care composition and/or deliver calcium ions to the oral cavity when the oral care composition is applied to the oral cavity. The calcium ion source can comprise a calcium salt, a calcium abrasive, and/or combinations thereof. In some cases, a calcium salt may also be considered a calcium abrasive or a calcium abrasive may also be considered a calcium salt.

The calcium ion source can comprise a calcium abrasive. The calcium abrasive can be any suitable abrasive compound that can provide calcium ions in an oral care composition and/or deliver calcium ions to the oral cavity when the oral care composition is applied to the oral cavity. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The calcium ion source can comprise a calcium salt, or a compound that can provide calcium ions in an oral care composition and/or deliver calcium ions to the oral cavity when the oral care composition is applied to the oral cavity that can not act as an abrasive. The calcium salt can comprise one or more calcium compounds, such as calcium chloride, calcium nitrate, calcium phosphate, calcium lactate, calcium oxalate, calcium oxide, calcium gluconate, calcium citrate, calcium bromide, calcium iodate, calcium iodide, hydroxyapatite, fluorapatite, calcium sulfate, calcium glycerophosphate, and/or combinations thereof.

The calcium ion source can have a iPUM-Ca of at least 2 times, at least 4 times, at least 6 times of the iPUM-Ca of untreated biofilm as described herein.

The oral care composition can comprise from about 5% to about 70%, from about 10% to about 50%, from about 10% to about 60%, from about 20% to about 50%, from about 25% to about 40%, or from about 1% to about 50% of a calcium ion source.

### Buffering Agent

The oral care composition can comprise a buffering agent. The buffering agent can be a weak acid or base that can maintain a particular pH at a selected site in the oral cavity. For example, the buffering agent can maintain a pH at a tooth's surface to mitigate the impact of plaque acids produced by bacteria. The buffering agent can comprise a conjugate acid of an ion also present in the oral care composition. For example, if the calcium ion source comprises calcium carbonate, the buffering agent can comprise a bicarbonate anion (—HCO₃⁻). The buffering agent can comprise a conjugate acid/base pair, such as citric acid and sodium citrate.

Suitable buffering systems can include phosphate, citrate salts, carbonate/bicarbonate salts, a tris buffer, imidazole, urea, borate, and/or combinations thereof. Suitable buffering agents include bicarbonate salts, such as sodium bicarbonate, glycine, orthophosphate, arginine, urea, and or/combinations thereof.

The oral care composition can comprise from about 0.5% to about 30%, from about 5% to about 25% or from about 10% to about 20%, of one or more buffering agents.

Biofilm Modifier The oral care composition can comprise one or more biofilm modifiers. A biofilm modifier can comprise a polyol, an ammonia generating compound, and/or a glucosyltransferase inhibitor.

A polyol is an organic compound with more than one hydroxyl functional groups. The polyol can be any suitable compound that can weakly associate, interact, or bond to tin ions while the oral care composition is stored prior to use. The polyol can be a sugar alcohol, which area class of polyols that can be obtained through the hydrogenation of sugar compounds with the formula (CHOH)ₙH₂. The polyol can be glycerin, erythritol, xylitol, sorbitol, mannitol, butylene glycol, lactitol, and/or combinations thereof. The oral care composition can comprise 0.01% to about 70%, from about 5% to about 70%, from about 5% to about 50%, from about 10% to about 60%, from about 10% to about 25%, or from about 20% to about 80%, by weight of the oral care composition, of a polyol.

The ammonia generating compound can be any suitable compound that can generate ammonia upon delivery to the oral cavity. Suitable ammonia generating compounds include arginine, urea, and/or combinations thereof. The oral care composition can comprise from about 0.01% to about 10%, from about 1% to about 5%, or from about 1% to about 25% of one or more ammonia generating compounds.

The glucosyltransferase inhibitor can be any suitable compound that can inhibit a glucosyltransferase. Glucosyltransferases are enzymes that can establish natural glycosidic linkages. In particular, these enzymes break down poly- or oligosaccharide moieties into simple sugars for bacteria associated with dental caries. As such, any compound that can inhibit this process can help prevent dental caries. Suitable glucosyltransferase inhibitors include oleic acid, epicatechin, tannins, tannic acid, moenomycin, caspofungin, ethambutol, lufenuron, and/or combinations thereof. The oral care composition can comprise from about 0.001% to about 5%, from about 0.01% to about 2%, or about 1% of one or more glucosyltransferase inhibitors.

### Metal Ion Source

The oral care composition can comprise metal, such as from a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The trace metal source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of a metal ion source.

### Bioactive Materials

The oral care composition can also include bioactive materials suitable for the remineralization of a tooth. Suitable bioactive materials include bioactive glasses, Novamin™, Recaldent™, hydroxyapatite, one or more amino acids, such as, for example, arginine, citrulline, glycine, lysine, or histidine, or combinations thereof. Suitable examples of compositions comprising arginine are found in U.S. Pat. Nos. 4,154,813 and 5,762,911, which are herein incorporated by reference in their entirety. Other suitable bioactive materials include any calcium phosphate compound. Other suitable bioactive materials include compounds comprising a calcium source and a phosphate source.

Amino acids are organic compounds that contain an amine functional group, a carboxyl functional group, and a side chain specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutonic acid, diaminoproprionic acid, salts thereof, and/or combinations thereof.

Bioactive glasses are comprising calcium and/or phosphate which can be present in a proportion that is similar to hydroxyapatite. These glasses can bond to the tissue and are biocompatible. Bioactive glasses can include a phosphopeptide, a calcium source, phosphate source, a silica source, a sodium source, and/or combinations thereof.

The oral care composition can comprise from about 0.01% to about 20%, from about 0.1% to about 10%, or from about 1% to about 10% of a bioactive material by weight of the oral care composition.

### Abrasive

The oral care composition can comprise a calcium abrasive, as described herein, and/or a non-calcium abrasive, such as bentonite, silica gel (by itself, and of any structure), precipitated silica, amorphous precipitated silica (by itself, and of any structure as well), hydrated silica, perlite, titanium dioxide, calcium pyrophosphate, dicalcium phosphate dihydrate, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. Such materials can be introduced into the oral care compositions to tailor the polishing characteristics of the target dentifrice formulation. The oral care composition can comprise from about 5% to about 70%, from about 10% to about 50%, from about 10% to about 60%, from about 20% to about 50%, from about 25% to about 40%, or from about 1% to about 50%, by weight of the oral care composition, of the non-calcium abrasive.

Alternatively, the oral care composition can be substantially free of, essentially free of, or free of silica, alumina, or any other non-calcium abrasive. The oral care composition can comprise less than about 5%, less than about 1%, less than about 0.5%, less than about 0.1%, or 0% of a non-calcium abrasive, such as silica and/or alumina.

### Water

The oral care composition of the present invention can be anhydrous, a low water formulation, or a high water formulation. In total, the oral care composition can comprise from 0% to about 99%, from about 5% to about 75%, about 20% or greater, about 30% or greater, or about 50% or greater by weight of the composition, of water. Preferably, the water is USP water.

In a high water oral care composition and/or toothpaste formulation, the oral care composition comprises from about 45% to about 75%, by weight of the composition, of water. The high water oral care composition and/or toothpaste formulation can comprise from about 45% to about 65%, from about 45% to about 55%, or from about 46% to about 54%, by weight of the composition, of water. The water may be added to the high water formulation and/or may come into the composition from the inclusion of other ingredients.

In a low water oral care composition and/or toothpaste formulation, the oral care composition comprises from about 5% to about 45%, by weight of the composition, of water. The low water oral care composition can comprise from about 5% to about 35%, from about 10% to about 25%, or from about 20% to about 25%, by weight of the composition, of water. The water may be added to the low water formulation and/or may come into the composition from the inclusion of other ingredients.

In an anhydrous oral care composition and/or toothpaste formulation, the oral care composition comprises less than about 10%, by weight of the composition, of water. The anhydrous composition comprises less than about 5%, less than about 1%, or 0%, by weight of the composition, of water. The water may be added to the anhydrous formulation and/or may come into the composition from the inclusion of other ingredients.

A mouth rinse formulation comprises from about 75% to about 99%, from about 75% to about 95%, or from about 80% to about 95% of water.

The composition can also comprise other orally acceptable carrier materials, such as alcohol, humectants, polymers, surfactants, and acceptance improving agents, such as flavoring, sweetening, coloring and/or cooling agents.

pH

The pH of the disclosed composition can be from about 4 to about 10, from about 7 to about 10, greater than 7 to about 10, greater than 8 to about 10, greater than 7, greater than 7.5, greater than 8, greater than 9, or from about 8.5 to about 10.

### Zinc Ion Source

The oral care composition can comprise zinc, such as from a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc ion source may be present in the total oral care composition at an amount of from about 0.01% to about 10%, from about 0.2% to about 1%, from about 0.5% to about 1.5%, or from about 0.3% to about 0.6%, by weight of the dentifrice composition.

### Polyphosphates

The oral care composition can comprise polyphosphate, such as from a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO (XPO3),X, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of, essentially free of, or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about] to about 20%, or about 10% or less, by weight of the oral care composition, of the polyphosphate source.

### Humectants

The oral care composition can comprise one or more humectants, have low levels of a humectant, be substantially free of, substantially free of, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants.

Suitable anionic surfactants include, for example, the water soluble salts of alkyl sulfates having from 8 to 20 carbon atoms in the alkyl radical and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants include sarcosinates, such as sodium lauroyl sarcosinate, taurates, sodium lauryl sulfoacetate, sodium lauroyl isethionate, sodium laureth carboxylate, and sodium dodecyl benzene sulfonate. Combinations of anionic surfactants can also be employed.

Another suitable class of anionic surfactants are alkyl phosphates. The surface active organophosphate agents can have a strong affinity for enamel surface and have sufficient surface binding propensity to desorb pellicle proteins and remain affixed to enamel surfaces. Suitable examples of organophosphate compounds include mono-, di- or triesters represented by the general structure below wherein Z1, Z2, or Z3 may be identical or different with at least one being an organic moiety. Z1, Z2, or Z3 can be selected from linear or branched, alkyl or alkenyl group of from 1 to 22 carbon atoms, optionally substituted by one or more phosphate groups; alkoxylated alkyl or alkenyl, (poly) saccharide, polyol or polyether group.

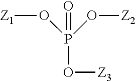

Some other agents include alkyl or alkenyl phosphate esters represented by the following structure:

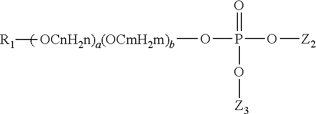

wherein R₁ represents a linear or branched, alkyl or alkenyl group of from 6 to 22 carbon atoms, optionally substituted by one or more phosphate groups; n and m, are individually and separately, 2 to 4, and a and b, individually and separately, are 0 to 20; Z and Z may be identical or different, each represents hydrogen, alkali metal, ammonium, protonated alkyl amine or protonated functional alkylamine, such as analkanolamine, or a R—(OCH₂)(OCH)— group. Examples of suitable agents include alkyl and alkyl (poly) alkoxy phosphates such as lauryl phosphate; PPGS ceteareth-10 phosphate; laureth-1 phosphate; laureth-3 phosphate; laureth-9 phosphate; trilaureth-4 phosphate; C₁₂₋₁₈ PEG 9 phosphate; and sodium dilaureth-10 phosphate. The alkyl phosphate can be polymeric. Examples of polymeric alkyl phosphates include those containing repeating alkoxy groups as the polymeric portion, in particular 3 or more ethoxy, propoxy isopropoxy or butoxy groups. Other suitable anionic surfactants are sarcosinates, isethionates and taurates, especially their alkali metal or ammonium salts. Examples include: lauroyl sarcosinate, myristoyl sarcosinate, palmitoyl sarcosinate, stearoyl sarcosinate oleoyl sarcosinate, or combinations thereof. Other suitable anionic surfactants include sodium or potassium alkyl sulfates, such as sodium lauryl sulfate, acyl isethionates, acyl methyl isethionates, alkyl ether carboxylates, acyl alaninates, acyl gulatames, acyl glycinates, acyl sarconsinates, sodium methyl acyl taurates, sodium laureth sulfosuccinates, alpha olefin sulfonates, alkyl benze sulfonates, sodium lauroyl lactylate, sodium laurylglucosides hydroxypropyl sulfonate, and/or combinations.

Zwitterionic or amphoteric surfactants useful herein include derivatives of aliphatic quaternary ammonium, phosphonium, and Sulfonium compounds, in which the aliphatic radicals can be straight chain or branched, and one of the aliphatic substituents contains from 8 to 18 carbon atoms and one contains an anionic water-solubilizing group, e.g., carboxy, sulfonate, sulfate, phosphate or phosphonate. Suitable betaine surfactants are disclosed in U.S. Pat. No. 5,180,577. Typical alkyl dimethyl betaines include decyl betaine or 2-(N-decyl-N,N-dimethylammonio)acetate, coco-betaine or 2-(N-coco-N,N-dimethyl ammonio)acetate, myristyl betaine, palmityl betaine, lauryl betaine, cetyl betaine, cetyl betaine, stearyl betaine, etc. The amidobetaines can be exemplified by cocoamidocthyl betaine, cocoamidopropyl betaine (CADB), and lauramidopropyl betaine. Other suitable amphoteric surfactants include betaines, sultaines, sodium laurylamphoacetates, alkylamphodiacetates, and/or combinations thereof.

Cationic surfactants useful in the present invention include, for example, derivatives of quaternary ammonium compounds having one long alkyl chain containing from 8 to 18 carbon atoms such as lauryl trimethylammonium chloride; cetyl pyridinium chloride; cetyl trimethyl-ammonium bromide; cetyl pyridinium fluoride or combinations thereof.

Nonionic surfactants that can be used in the compositions of the present invention include, for example, compounds produced by the condensation of alkylene oxide groups (hydrophilic in nature) with an organic hydrophobic compound which may be aliphatic or alkylaromatic in nature. Examples of suitable nonionic surfactants can include the Pluronics® which are poloxamers, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and combinations of such materials. Other suitable non-ionic surfactants includes alkyl glucamides, alkyl glucosides, and/or combinations thereof.

The one or more surfactants can also include one or more natural and/or naturally derived surfactants. Natural surfactants can include surfactants that are derived from natural products and/or surfactants that are minimally or not processed. Natural surfactants can include hydrogenated, non-hydrogenated, or partially hydrogenated vegetable oils, olus oil, Passiflora incarnata oil, candelilla cera, coco-caprylate, caprate, dicaprylyl ether, lauryl alcohol, myristyl myristate, dicaprylyl ether, caprylic acid, caprylic ester, octyl decanoate, octyl octanoate, undecane, tridecane, decyl oleate, oleic acid decylester, cetyl palmitate, stearic acid, palmitic acid, glyceryl stearate, hydrogenated, non-hydrogenated, or partially hydrogenated vegetable glycerides, Polyglyceryl-2 dipolyhydroxystearate, cetearyl alcohol, sucrose polystearate, glycerin, octadodecanol, hydrolyzed, partially hydrolyzed, or non-hydrolyzed vegetable protein, hydrolyzed, partially hydrolyzed, or non-hydrolyzed wheat protein hydrolysate, polyglyceryl-3 diisostearate, glyceryl oleate, myristyl alcohol, cetyl alcohol, sodium cetearyl sulfate, cetearyl alcohol, glyceryl laurate, capric triglyceride, coco-glycerides, lectithin, dicaprylyl ether, xanthan gum, sodium coco-sulfate, ammonium lauryl sulfate, sodium cocoyl sulfate, sodium cocoyl glutamate, polyalkylglucosides, such as decyl glucoside, cetearyl glucoside, cetyl stearyl polyglucoside, coco-glucoside, and lauryl glucoside, and/or combinations thereof. Natural surfactants can include any of the Natrue ingredients marketed by BASF, such as, for example, CegeSoft®, Cetiol®, Cutina®, Dehymuls®, Emulgade®, Emulgin®, Eutanol®, Gluadin®, Lameform®, LameSoft®, Lanette®, Monomuls®, Myritol®, Plantacare®, Plantaquat®, Platasil®, Rheocare®, Sulfopon®, Texapon®, and/or combinations thereof.

Other specific examples of surfactants include sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Additional surfactants desired include fatty acid salts of glutamate, alkyl glucoside, salts of taurates, betaines, caprylates, and/or mixtures thereof. The oral care composition can also be sulfate free.

The oral care composition can comprise one or more surfactants each at a level from about 0.01% to about 15%, from about 0.3% to about 10%, or from about 0.3% to about 2.5%, by weight of the oral care composition.

### Thickening Agents

The oral care composition can comprise one or more thickening agents. Thickening agents can be useful in the oral care compositions to provide a gelatinous structure that stabilizes the dentifrice and/or toothpaste against phase separation. Suitable thickening agents include polysaccharides, polymers, and/or silica thickeners.

The thickening agent can comprise one or more polysaccharides. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, microcrystalline cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

Other polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota caragcenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. Alternatively, the oral care composition can be free of, substantially free of, or essentially free of a copolymer of maleic anhydride and methyl vinyl ether.

The thickening agent can comprise one or more inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.1% to about 10%, from about 0.2% to about 5%, or from about 0.5% to about 2% of one or more thickening agents.

### Prenylated Flavonoids

The oral care composition of the present invention can comprise prenylated flavonoid. Flavonoids are a group of natural substances found in a wide range of fruits, vegetables, grains, bark, roots, stems, flowers, tea, and wine. Flavonoids can have a variety of beneficial effects on health, such as antioxidative, anti-inflammatory, antimutagenic, anticarcinogenic, and antibacterial benefits. Prenylated flavonoids are flavonoids that include at least one prenyl functional group (3-methylbut-2-en-1-yl, as shown in Formula VIII), which has been previously identified to facilitate attachment to cell membranes. Thus, while not wishing to being bound by theory, it is believed that the addition of a prenyl group, i.e. prenylation, to a flavonoid can increase the activity of the original flavonoid by increasing the lipophilicity of the parent molecule and improving the penetration of the prenylated molecule into the bacterial cell membrane. Increasing the lipophilicity to increase penetration into the cell membrane can be a double-edged sword because the prenylated flavonoid will tend towards insolubility at high Log P values (high lipophilicity). Log P can be an important indicator of antibacterial efficacy.

As such, the term prenylated flavonoids can include flavonoids found naturally with one or more prenyl functional groups, flavonoids with a synthetically added prenyl functional group, and/or prenylated flavonoids with additional prenyl functional groups synthetically added.

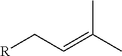

Formula VIII. Prenyl Function Group with R representing the other portions of the molecule

Other suitable functionalities of the parent molecule that improve the structure-activity relationship (e.g., structure-MIC relationship) of the prenylated molecule include additional heterocycles containing nitrogen or oxygen, alkylamino chains, or alkyl chains substituted onto one or more of the aromatic rings of the parent flavonoid.

Flavonoids can have a 15-carbon skeleton with at least two phenyl rings and at least one heterocyclic ring. Some suitable flavonoid backbones can be shown in Formula IX (flavone backbone), Formula X (isoflavan backbone), and/or Formula XI (neoflavonoid backbone).

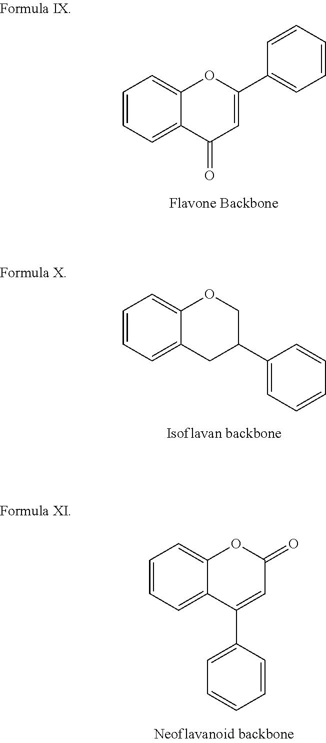 [SMILES: O=c1cc(-c2ccccc2)oc2ccccc12 ; c1ccc(C2COc3ccccc3C2)cc1 ; O=c1cc(-c2ccccc2)c2ccccc2o1]

Other suitable subgroups of flavonoids include anthocyanidins, anthoxanthins, flavanones, flavanonols, flavans, isoflavonoids, chalcones and/or combinations thereof.

Prenylated flavonoids can include naturally isolated prenylated flavonoids or naturally isolated flavonoids that are synthetically altered to add one or more prenyl functional groups through a variety of synthetic processes that would be known to a person of ordinary skill in the art of synthetic organic chemistry.

Other suitable prenylated flavonoids can include Bavachalcone, Bavachin, Bavachinin, Corylifol A, Epimedin A, Epimedin A1, Epimedin B, Epimedin C, Icariin, Icariside I, Icariside II, Icaritin, Isobavachalcone, Isoxanthohumol, Neobavaisoflavone, 6-Prenylnaringenin, 8-Prenylnaringenin, Sophoraflavanone G, (-)-Sophoranone, Xanthohumol, Quercetin, Macelignan, Kuraridin, Kurarinone, Kuwanon G, Kuwanon C, Panduratin A, 6-geranylnaringenin, Australone A, 6,8-Diprenyleriodictyol, dorsmanin C, dorsmanin F, 8-Prenylkaempferol, 7-O-Methylluteone, luteone, 6-prenylgenistein, isowighteone, lupiwighteone, and/or combinations thereof. Other suitable prenylated flavonoids include cannflavins, such as Cannflavin A, Cannflavin B, and/or Cannflavin C.

Preferably, the prenylated flavonoid has a high probability of having an MIC of less than about 25 ppm for S. aureus, a gram-positive bacterium. Suitable prenylated flavonoids include Bavachin, Bavachinin, Corylifol A, Icaritin, Isoxanthohumol, Neobavaisoflavone, 6-Prenylnaringenin, 8-Prenylnaringenin, Sophoraflavanone G, (-)-Sophoranone, Kurarinone, Kuwanon C, Panduratin A, and/or combinations thereof.

Preferably, the prenylated flavonoid has a high probability of having an MIC of less than about 25 ppm for E. coli, a gram-negative bacterium. Suitable prenylated flavonoids include Bavachinin, Isoxanthohumol, 8-Prenylnaringenin, Sophoraflavanone G, Kurarinone, Panduratin A, and/or combinations thereof.

Approximately 1000 prenylated flavonoids have been identified from plants. According to the number of prenylated flavonoids reported before, prenylated flavonones are the most common subclass and prenylated flavanols is the rarest sub-class. Even though natural prenylated flavonoids have been detected to have diversely structural characteristics, they have a narrow distribution in plants, which are different to the parent flavonoids as they are present almost in all plants. Most of prenylated flavonoids are found in the following families, including Cannabaceae, Guttiferae, Leguminosae, Moraceae, Rutaceae and Umbelliferae. Leguminosae and Moraceae, due to their consumption as fruits and vegetables, are the most frequently investigated families and many novel prenylated flavonoids have been explored. Humulus lupulus of the Cannabaceae include 8-prenylnaringenin and xanthohumol, which play an important role in the health benefits of beer.

The prenylated flavonoid can be incorporated through the hops extract, incorporated in a separately added extract, or added as a separate component of the oral care compositions disclosed herein.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO2, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### F-Content in the Slurry by ADA One-Minute Release

The fluoride content in the rat caries treatment slurries can be estimated using the ADA One-Minute Release method. The exact methodology is maintained by the ADA as part of its seal acceptance program and is available from the Association. An outline of the method as used herein is described below. The ADA also references the ANSI/ADA Standard No. 116 Oral Rinses or ISO 16408 Dentistry-Oral Hygiene Products-Oral Rinses for an example ion-selective electrode method for determining the ionic fluoride content in oral care products.

A stock fluoride solution (0.5 mg/mL, 500 ppm, Ricca, RC3172-16, Arlington, TX) was diluted to make solutions of 5 ppm, 25 ppm, 50 ppm, and 250 ppm using deionized water. Each standard was diluted at a 1:1 ratio with TISAB II (Ricca, Arlington, TX) buffer solution to create calibration solutions (TISAB IV (Ricca, Arlington, TX) if the calibration solutions are intended for stannous fluoride samples).

A calibration curve was created using the calibration solutions. 200 μL of 25 ppm calibration solution was placed in a microsample cup. A fluoride electrode (VWR, Radnor, PA) was placed in the solution and the mV of each sample is noted by reading the electrode meter (VWR, Radnor, PA). The procedure was repeated for each prepared calibration solution. A calibration curve was constructed by plotting mV vs. Log [F-].

Paste dentifrice samples were prepared by weighing approximately 4 g of each paste sample in a 70 mL speed mixer cup. Next, 12 mL of deionized was added to the sample cup. The speed mixer cup was mixed for 60 seconds (FlackTek Speedmixer, Landrum, SC) using Speedmixer Program #7, which is 800 rpm for 5 seconds and then 2200 rpm for 55 seconds. The slurry samples were transferred into a centrifuge tube and then centrifuged for 10 minutes at 11,000 rpm.

Samples were prepared for analysis by combining 1 mL of the supernatant and 1 mL of fresh TISAB II buffer for sodium fluoride samples and TISAB IV for stannous fluoride samples. The samples were quickly mixed using a vortex mixer. 200 μL of the solution were transferred to a micro sample cup. The fluoride electrode was placed in the cap. The value (mV) was recorded.

Values for released fluoride were calculated using Formula 1, provided below:

$$ADA Fluoride Released = Average [ F ] ppm \times Dilution Factor \times 100 \qquad Formula 2$$

$$[ F ] = Fluoride Concentration of Sample Supernatant ( ppm )$$

$$Dilution Factor :$$

$$NaF and {SnF}_{2} = 4 - ( Formula \% Insoluable Raw Materials / 100 )$$

$$* Insoluable Raw Materials ( IRM ) include silica , titanium dioxide , mica , and prills .$$

For historical samples for which the slurry fluoride content was not recorded, the ADA one-minute fluoride release was estimated based on the recorded formulated fluoride content, fluoride source, and abrasive combination. In some instances, the formulated fluoride content was used when it is known to closely correspond to the ADA one-minute fluoride release results, i.e., for Crest® Cavity Protection.

For MFP-containing toothpastes, additional steps were needed to liberate the fluoride ion such that it could be measured by an ion selective electrode. The steps were the same up to isolation of the supernatant. Following supernatant isolation, a 1.5 mL aliquot of the MFP-containing supernatant was combined with 2.5 mL of 2N hydrochloric acid (VWR, Radnor, PA) in an airtight tube that was caped tightly and mixed vigorously for 30 seconds. The tube headspace was minimized as much as possible. It was then heated to 50° C. for 20 minutes and cooled to room temperature at ambient, benchtop conditions. Once it was cool, 2.575 mL of 2N NaOH (VWR, Radnor, PA) was added to the airtight tube, capped, and mixed vigorously for 30 seconds. The resulting solution was allowed again to cool to room temperature. An aliquot of this was buffered as described above with TISAB II and measured as described above using a fluoride ion selective electrode. The appropriate corrections for dilution were made when determining the ADA fluoride released value.

Successful demonstration of an analyst's ability to execute the method can be demonstrated by achieving an ADA fluoride released between about 95% to 100% for the USP NaF/Silica toothpaste or for about 70% to about 85% for the USP SnF2/Silica toothpaste. The coefficient of correlation for the F calibration curve on the ion selective electrode should be 0.995 or higher. Verify the accuracy by running check standards that bracket the results before and after sample readings.

### Sn-Free In Vitro Plaque Glycolysis and Regrowth Method

The in vitro plaque glycolysis model (iPGRM) is a technique in which plaque is grown from human saliva and treated with various agents to determine anti-glycolytic activity of treatments. When bacteria convert sugar into energy with the help of enzymes, acids are formed. These acids demineralize and damage the dental enamel. The purpose of this technique is to provide a simple method for determining if treatment compounds have an inhibitory effect on the metabolic pathways that plaque microorganisms utilize for the production of acids or toxins and/or inhibit their growth. For the purposes of the work here, if the test therapeutic compositions contain Sn, the Sn placebo should be tested or controlled for correctly. Additionally, the antibacterial composition should be tested with respect to its placebo to determine the iPGRM value for the antibacterial composition only. This is important if buffers, e.g., bicarbonate, orthophosphate, calcium carbonate, are present in the composition in addition to the antibacterial composition.

A plaque biofilm was grown on glass rods from fresh pooled human saliva and Trypticase Soy Broth (TSB) at 37° C. over 2 days by dipping glass rods into and out of media in a reciprocating motion using a rack suspended on a rod and moved by a reciprocating motor inside of an incubation oven. Treatments were 2 minutes of dentifrice slurry in water (1:5) or diluted treatment in water (1:5). After treatments, biofilms were incubated with TSB and sucrose until pH indicator showed a color change (˜ 6 hrs). The pH of the media solutions was then measured to determine the amount of glycolysis inhibition relative to a negative control.

Prior to Day 1, but within a week of Day 1, new glass rods (5 mm×90 mm) were polished approximately 25 mm from the un-tapered end on a lathe with silicon carbide paper of 240, 320, 400, and 600 grit used sequentially. After the initial polishing, the rods should be polished with 600 grit paper before each test. After polishing, rods were stored until ready to run. Enough rods should be polished for a full rack of treatments. A rack can treat 12 compositions with 4 replicates of each composition such that the rack has 48 rods.

On Day 1, saliva was collected daily during the test from a panel of 5-10 people by paraffin stimulation and was refrigerated at 4° C. until it was needed throughout the day. Pool saliva carefully (do not pour in wax/mucus) and mix thoroughly before use. Saliva should be collected from panelists free from disease, who are not on any antibacterial medication or medication that modifies saliva flow, and who brush regularly with Crest Cavity Protection. The rods were removed from storage, rinsed with deionized water to remove any sanding residue, disinfected in 70% ethanol/water solution, and were allowed to dry on a sterile surface. Subsequently, the rods were loaded into a hanging rack of holders that were used to dip the rods continuously into media vials containing growth media. The rod heights were adjusted and each rod was secured in place using a rubber o-ring. In the early afternoon, 7 mL of growth media (160 g of a solution of 3% TSB (VWR, Radnor, PA) with 3% sucrose (VWR, Radnor, PA) was mixed with 240 g pooled human saliva. This TSB/sucrose solution should be sterilized by autoclave before combining with the pooled human saliva.) into media vials. The media vials were arranged under the hanging rods on a rack in an incubation oven. The incubator has been previously modified such that a dipping motor can dip the rods into the media vials submerging 1.5 cm of the rod into the growth media at a frequency of 1 dip per minute without the rods touching the walls of the media vial. The rods were dipped overnight this way.

On Day 2, an enriched growth media was prepared (500 g of a solution of 3% TSB and 10% sucrose was mixed with 33 g pooled human saliva. This TSB/sucrose solution should be sterilized by autoclave before combining with the pooled human saliva.). This enriched growth media was pipetted into a new set of media vials (7 mL per vial) and was swapped for the overnight growth media from Day 1. The rods were dipped throughout the day in this enriched growth media for 5 hours at 37° C. in the incubation oven. At the end of the day, a new overnight growth media was prepared (40 g of a solution of 3% TSB was mixed with 360 g pooled human saliva and 0.5 g sucrose), pipetted into a new set of media vials, and swapped for the enriched growth media. The rods were dipped overnight in the same fashion as on the first day.

On Day 3, a glycolysis media was prepared by combining 0.15 g TSB, 25 g sucrose, and 500 mL deionized water resulting in a solution of 0.03% TSB and 0.5% sucrose in water. This solution was mixed then sterilized in an autoclave. The pH was then adjusted to 6.5 using 0.1M HCl and pipetted into new media vials (7 mL). Two extra vials were filled than were needed for the rack of rods as pH blanks. Two drops of chlorophenol red solution were added to each of the 4 tubes that contained the glycolysis media for the negative control treatment group (Crest Cavity Protection slurry). Three drops of bromocresol purple solution were added to 2 tubes that contained the glycolysis media for the positive control treatment group (1% Chlorhexidine solution). Set the rack aside until treatments are complete. Two sets of vials were prepared containing 12 mL of deionized water to rinse off the treatments. Vials were prepared containing the treatment slurries/solutions (7 mL) of homogenized treatment and water. The rods were dipped into the treatment vials for 2 minutes, rinsed for 10 dips in a first set rinse vials, rinsed for 10 dips in a second set of rinse vials, rinsed for 10 dips in a third set of rinse vials, and returned to the incubator rack. It is very important that the biofilm on the rods not touch the walls of any of the media, treatment, or rinse vials that would result in the removal of biofilm. The entire biofilm was treated and rinsed. Once all treatments were complete, the biofilms on the rods were fully submerged in the glycolysis media inside the incubation oven with no dipping for 2 hours. After two hours, the dipping apparatus was activated. The total incubation time was between 3 to 7 hours. Incubation is terminated when the pH value in the glycolysis media of the negative controls is between 4.8-5.6, more ideally 4.9-5.2, and when the pH value in the glycolysis media of the positive controls is above the negative control. If the indicator dye in the positive control turns yellow, i.e., the pH has dropped beneath 5.2, the incubation has gone on too long and the test will need to be repeated.

After incubation termination on Day 3, the rods were removed from the glycolysis media and allowed to dry in the oven. The glycolysis media was removed from the incubation oven, allowed to return to room temperature, and the pH was measured in each vial and the blank vials to determine the average pH change of the media following treatment. The change in pH is determined with respect to the blank vials. If the final pH of the blank is less than 6.6, the test needs to be repeated. If the difference between the positive and negative control is not significant in a student's t-test, the test needs to be repeated. If the change in pH of the negative control of the negative control with respect to the blank is less than 1, the test needs to be repeated.

After the pH values of all the vials were measured, the ΔpH per vial was determined by subtracting its pH from the average pH of the blanks. The glycolysis inhibition efficacy is determined from the following formula. The average ΔpH of a treatment was determined by averaging the results from the four replicate vials per treatment.

$$Efficacy ( \% ) = 100 - ( \frac{Avg Δ {pH}_{sample}}{Avg Δ {pH}_{neg ctrl}} ) \times 100 \qquad Formula 3$$

If the efficacy of the positive control (1% Chlorhexidine solution) is not between about 65% to about 85% with respect to the negative control (Crest Cavity Protection, Procter & Gamble, Cincinnati, OH), the test needs to be repeated.

### In Vitro Plaque Uptake Method for Calcium

The in vitro plaque uptake model (iPUM) is a technique in which plaque is grown from thawed form frozen human saliva and treated with various agents to determine the uptake of elemental components into the plaque. A plaque biofilm was grown on glass rods from frozen pooled human saliva and Trypticase Soy Broth (TSB) at 37° over 3 days by dipping glass rods into and out of media in a reciprocating motion. Treatments were 2 minutes of dentifrice slurry in sterile water (1:5). After treatments, biofilms are dried, weighed, digested and analyzed by either ISE (fluoride) or ICP-OES (Ca, Sn, Zn, etc.).

Prior to Day 1, but within a week of Day 1, new glass rods (5 mm×90 mm) were polished approximately 25 mm from the un-tapered end on a lathe with silicon carbide paper of 240, 320, 400, and 600 grit used sequentially. After the initial polishing, the rods should be polished with 600 grit paper before each test. After polishing, rods were stored until ready to run test. Enough rods should be polished for a full rack of treatments. A rack can treat 12 compositions with 4 replicates of each composition such that the rack has 48 rods. Saliva was collected daily from a panel of 5-10 people by paraffin stimulation and frozen overnight. Pool saliva carefully (do not pour in wax/mucus) and mix thoroughly before use. Enough saliva should be frozen prior to the study starting to last the entire study. Saliva should be collected from panelists free from disease, who are not on any antibacterial medication or medication that modifies saliva flow, and who brush regularly with Crest Cavity Protection.

On Day 1, the rods were removed from storage, rinsed with deionized water to remove any sanding residue, disinfected in 70% ethanol/water solution, and were allowed to dry on a sterile surface. Subsequently, the rods were loaded into a hanging rack of holders that were used to dip the rods continuously into media vials containing growth media. The rod heights were adjusted and each rod was secured in place using a rubber o-ring. In the early afternoon, 7 mL of growth media (160 g of a solution of 3% TSB (VWR, Radnor, PA) with 3% sucrose (VWR, Radnor, PA) was mixed with 240 g thawed pooled human saliva. This TSB/sucrose solution should be sterilized by autoclave before combining with the pooled human saliva. Frozen saliva can be thawed in the refrigerator or in a bucket of warm, not hot, water) into media vials. The media vials were arranged under the hanging rods on a rack in an incubation oven. The incubator has been previously modified such that a dipping motor can dip the rods into the media vials submerging 1.5 cm of the rod into the growth media at a frequency of 1 dip per minute without the rods touching the walls of the media vial. The rods were dipped overnight this way.

On Day 2 and Day 3, an enriched growth media was prepared (500 g of a solution of 3% TSB and 10% sucrose was mixed with 33 g thawed pooled human saliva. This TSB/sucrose solution should be sterilized by autoclave before combining with the pooled human saliva.). This enriched growth media was pipetted into a new set of media vials (7 mL per vial) and was swapped for the overnight growth media. The rods were dipped throughout the day in this enriched growth media for 5 hours at 37° C. in the incubation oven. At the end of the day, a new overnight growth media was prepared (40 g of a solution of 3% TSB was mixed with 360 g pooled human saliva and 0.5 g sucrose), pipetted into a new set of media vials, and swapped for the enriched growth media. The rods were dipped overnight in the same fashion as on the first day.

On Day 4, treatment slurries were prepared by homogenizing 4 g of treatment composition with 20 g deionized water and pipetted into treatment vials (7 mL) around such that the rods can be dipped into them. If the slurries were prepared ahead of time, they should be agitated via pipette immediately prior to treatment to ensure good suspension of solid sources of calcium. Two sets of vials were prepared containing 12 mL of deionized water to rinse off the treatments. The rods were dipped into the treatment vials for 2 minutes, rinsed for 10 dips in a first set rinse vials, rinsed for 10 dips in a second set of rinse vials, rinsed for 10 dips in a third set of rinse vials, and returned to the incubator rack. The entire biofilm was treated and rinsed. Once all treatments were complete, the biofilms on the rods were returned to the incubator oven to dry. It is very important that the biofilm on the rods not touch the walls of any of the media, treatment, or rinse vials that would result in the removal of biofilm.

The final analysis for calcium occured after the rods were weighed to determine their weight with biofilm, digesting the rods at 90° C. in concentrated nitric acid (BDH, Radnor, PA, Aristar Plus for trace metal analysis) in a 50 mL conical tube, removing the rods from the digest, cooling and diluting the digest to 50 mL with deionized water, and analyzing the digest for Ca via inductively coupled plasma-optical emission spectroscopy (ICP-OES) or inductively coupled plasma-mass spectrometry (ICP-MS) (Agilent, Santa Clara, CA). Rinsed and dried rods were weighed, and the biofilm mass was calculated by subtracting the biofilm-free rod mass from the rod mass with biofilm.

The total calcium mass in the biofilm was determined for each rod from the analysis of the digest and divided by the biofilm mass for that rod to determine the mass-normalized calcium uptake that was then averaged across the replicates. The ratio of the calcium uptake between an experimental treatment and that of Crest Cavity Protection was determined by dividing the mass-normalized calcium uptake for the experimental treatment averaged across the replicates by the mass-normalized calcium uptake for the Crest Cavity Protection treatment averaged across the replicates.

The mass-normalized calcium uptake for the Crest Cavity Protection group should be about 0.5-3 micro-gca/mgbiofilm. If not, the test should be repeated. The mass-normalized calcium uptake for Tom's of Maine Rapid Relief Sensitive (Tom's of Maine, Kennebunk, ME) should be about 2-3.5 times that of Crest Cavity Protection. If not, the test should be repeated.

### F-Free HAP Dissolution

The HAP dissolution method was designed to test the acid protection of a chosen test dentifrice. After treating hydroxyapatite powder (HAP) with test dentifrice slurries, the HAP is added to an acidic media and the change in pH is an indicator of the degree of surface adsorption. The smaller the pH rise, the better the surface protection.

Dentifrice slurries (1:3 paste: water) were prepared for the experiment. Specifically, 10 g of dentifrice paste was combined with 30 g of deionized water in a 50 mL container with a stir bar. The container was placed on a stir plate to mix until the two components were mixed. The paste slurries were centrifuged at 15,000 rpm for 15 min to isolate their supernatant.

For each treatment, including for the water control, 0.300 g of hydroxyapatite powder (HAP) was placed into a 50 mL round bottom centrifuge tube. For treatment with a dentifrice paste, 24 mL of the prepared dentifrice supernatant was added to the HAP. Each treated HAP sample was immediately vortex mixed (DVX-2500 multi-tube vortexer, VWR, Radnor, PA) at 2500 rpm for 2 minutes. All samples were then centrifuged at 15,000 rpm for 15 minutes. The liquid phase was decanted out of the centrifuge tube, which left a HAP pellet. The remaining HAP pellet was rinsed three times by adding deionized water, vortex mixing at 2500 rpm for 1 minute, centrifuging at 15,000 rpm for 15 minutes, and the liquid phase was decanted out of the centrifuge tube. The treated HAP pellet was dried in a 55° C. oven overnight.

Samples of HAP were analyzed for ΔpH. 25 mL of 10 mM citric acid (1.9212 g of citric acid in 1 L of deionized water) was added to a 50 mL beaker with a stir bar. The beaker was placed on a stir plate (Metrohm, Herisau, Switerland, Model No. 728) and turned on. The Titrano pH electrode (Metrohm, Herisau, Switzerland, Model No. 719S) was placed in the stirring beaker with citric acid. After equilibration of the citric acid solution (until pH has a minimum change of 2.5+0.001 pH within 30 second), 50 mg of the dried HAP powder was added to the citric acid solution. The pH was recorded at 10 min. The % efficacy was determined by Formula I, below.

$$\% Efficacy vs . Water = \frac{Average Δ pH Water - Average Δ pH Treatment}{Average Δ pH Water} \qquad Formula 4$$

For compositions containing fluoride, the fluoride-free composition should be made in order to determine its fluoride-free HAP dissolution efficacy ensuring that the pH and counter ion content (Na, Sn, etc.) has been properly controlled. For historic samples, the HAP dissolution efficacy was estimated based on the reported or formulated Sn content of the sample using a previously existing relationship. This relationship is given in FIG. 4.

The method will have been run correctly when the pH change for the water treated control samples is between about 1.3 and 1.5 pH units at 10 minutes. The method should be practiced and repeated until this result is achieved. For guidance, a pH change for a 1100 ppm F as NaF treated control sample should be between about 0.9 and 1.1.

### Anti-Cavity Performance by Rat Caries, Historical Method

Two rat caries methods have been used in our laboratory over the past 60 years to establish anti-caries performance. The first described here is the Historical Method. The second described later is the Modern Method. The relative performance of actives is similar in the two methods and the same trends are observed; however, the calculated percent reduction in caries relative to the placebo requires minor adjustment. This conversion is achieved by subtracting 5.6% from the Historical Method scores to give their Modern Method equivalents.

The anti-cavity performance of oral care compositions can be substantiated in rats using several methods (Stookey, et al. Adv. Dent. Res. 9 (3): 198-207, 1995). All of the data presented here use methods disclosed as part of the FDA Method 37 in the anti-caries monograph. The example given here is how our historical data were collected. This method is further described in the following articles: Francis, MD, Arch. Oral Biol. 11:141-1489, 1966; Briner, W W and Francis, MD, Caries Res. 5:180-187, 1971; Donaldson, JD, White, WE, Briner, W W, and Cooley, WE, J. Dent. Res. 53:648-652, 1974.

Wistar rats were obtained from Harlan Industries, Inc., Cumberland, Ind. They were shipped via truck and arrived on Day 0 at 22-23 days of age. Animals were received in twenty litters of ten randomly sexed animals per litter, thus providing the 200 animals used in a standard rat caries study. One animal from each litter was then randomly allocated to one of the treatment groups and placed in a numbered stainless-steel, wire-bottom cate (litter mates occupy the same position in each group; e.g., all animals form litter #1 were allocated to the first cage of each treatment group). Animals were then weighed in and the weight was recorded in the lab book. Animals were fed cariogenic diet #469 ad libitum and deionized water ad libitum.

Treatment required the use of long-stem, cotton-tipped swabs. The swab is dipped into a slurry prepared with toothpaste diluted 1:1 with deionized water. This dilution was mixed on a stir-plate for five minutes prior to treatment application. With the rat's mouth held open by means of a stainless-steel retaining clamp, the dipped swab was brushed against the maxillary molars with a front-to-back stroke repeated six times. On the mandible, the swab was dipped into the treatment slurry and then rotated toward the check, thereby moving around the tongue to reach the mandible molars. Again, six rotations per mandible were required. This procedure is repeated on the opposite side of the mouth with a fresh quantity of toothpaste slurry. Treatment was applied twice daily, beginning the Day 1 after arrival, through Day 3. (Day 4, normally a Saturday, and Day 5, normally a Sunday were not treatment days.) Treatment resumed on Day 6 through Day 10 with the weekend off (Days 11 and 12), and again resumed on Day 13 through Day 17 with Days 18 and 19 off. The following week the animals were treated on Day 20 and Day 21, and a final body weight was obtained.

On Day 22, the day after the final treatment day, the animals were sacrificed by decapitation. The tongue was excised and the cheeks incised to the angle of the jaw. A tag with the animal's number (cage number) was attached to the snout of the animal with an 8-inch string. The mouth was propped open with a short piece of Tygon tubing. Once animals from the entire test were sacrificed, the heads were lowered into vats of 2% silver nitrate staining solution for one hour. Upon removal from the stain, the heads were rinsed in at least three changes of running tap water. The heads were then placed in aluminum foil baking pans, the bottom of the pan covered with tap water and the pan covered loosely with heavy-gauge aluminum foil.

After staining, the aluminum baking pans containing the heads were autoclaved at approximately 120° C. and 10 lbs. pressure for 35 minutes, after which the steam was turned off and the pans were allowed to stand for another 15 minutes before the heads were removed. After this procedure, the bones containing both the upper and lower molars for each rat could easily be lifted from the surrounding tissue and placed into the animal's pre-number plastic vial for future identification. These vials were left open for 24-36 hours to dry at room temperature and were then closed until they were to be sectioned.

Next all the vials for the test were arranged numerically, and microscope slides were made up with corresponding animal number (one per rat) and the study number attached. Considering one animal at a time, each quadrant was hemi-sectioned longitudinally, and each section was permanently mounted on the microscope slide. Each quadrant occupied the same position on the slide as in the animal's mouth (e.g., the right upper quadrant was mounted on the right upper corner of the slide).

Using a microscope at 30× magnification, 22 fissures and 24 smooth surfaces were graded per slide/animal. Each fissure was divided by an imaginary line through the middle of its bottom, and then each side of the fissure was assigned a severity grade. Since each quadrant was sectioned longitudinally, both halves of each quadrant are graded, and the most severe grade is recorded for each corresponding smooth surface of half fissure. In all there were 68 grades per slide/animal. The method of score lesion severity is as follows:
0-no stain in the enamel or dentin at site.
1-dark brown stain in enamel only.
2-dark brown stain in enamel extending to the dentin/enamel junction but no further.
3-stain through the enamel into the dentin.

At the beginning of a study one group of rats was sacrificed to obtain a mean zero-time severity score per animal. At the end of the study all treatment groups were sacrificed and graded to obtain a mean severity score per animal. Thus in computing the severity of caries in ach particular group, the 68 smooth surface and half fissure grades for each animal in the group were totaled. This number was then divided by the number of animals in the group to obtain the mean severity expressed as the mean number of hypomineralized areas (x HMA).

With all x̅ HMA scores tabulated, a percent reduction is calculated for each treatment group. This is done by subtracting the score of the test group from that of the water or placebo control group and then dividing by the water or placebo control group score. This number is expressed as a percent by multiplying by 100. Further statistical analysis was performed to determine significant differences among the groups if they existed. A standard analysis of variance was used. Treatments were ranked by a Newman-Keuls analysis. All historical method scores have been converted to Modern Method equivalent scores as part of establishing the rat caries meta-analysis.

### Anti-Cavity Performance by Rat Caries, Modern Method

The test design used here is similar to those found in the FDA Method #37 of the Fluoride Anti-Caries OTC Monograph. The major variations are the diet used (MIT 200 rather than #469), the caries score method (Keyes method rather than HMA), and treatment frequency. Experimental procedures were conducted according to the FDA regulations Part 58.

Using litters as a covariate, the use of between 50 and 58 (depending on the type of fluoride) animals per treatment group satisfies the most stringent power requirements of the ADA's Council on Dental Therapeutics 20% clinical difference between treatments at 80% power. However, we have been routinely using 40 animals per treatment group and both the ADA's CDT and the FDA have consistently accepted these tests. This requires initiating the study with 40 animals per group. Twenty-three (23) litters provided these animals. When studies are sized as such, treatment differences of approximately 16% have been found to be significant on occasion, thus is generally considered the cusp of clinical significance.

All protocols are reviewed and approved by the Institutional Animal Care and Use Committee prior to the receipt of animals.

The animals were weanling mixed-sex Sprague Dawley rats; weighing 29-53 grams. Due to the shipping schedule of the supplier, the dams were received with their entire birth litter. The litters were received when the pups were 6 days of age and litter size was reduced at 8 days of age to ten (10) pups per litter. Twenty-five (25) litters were purchased. The five extra litters were to allow for any mortality prior to stratification. Any unused animals were euthanized after the study stratification.

The litters were maintained in large solid-bottom (box-type) cages with dams until the pups were weaned at 18 days of age. Starting at 9 days of pup age, the dams were rotated daily among the litters until the pups were weaned at 18 days of age. The pups were maintained in the box cages until 21 days of age. At that time, the pups were stratified and housed in pairs in suspended wire-bottomed cages that had been cleaned and sanitized prior to usage. The change in caging was required to prevent artificially increasing the caries rate due to direct contact bedding. The cages were arranged so that all animals of the individual groups were together and the cages were labeled with group designation and treatment (treatment code) that the animals received.

When the pups were 21 days of age they were given unique numbers by car-punch with records kept of littermates. Animals were assigned to groups in such a manner that groups were balanced for litter, weight and sex. There were 40 animals per group.

Upon receipt, dams and litters were provided rodent lab diet until the pups were 8 days of age. On day 8 (pup age) dams and litters were provided Diet MIT 305. Pups were provided Diet MIT 200 ad libitum at day 18 (pup age) and throughout the test period. All animals were provided with deionized water ad libitum.

Box caging was changed at day 13 and again at day 18 of pup age. Following administration of the inoculum, box cages and the bedding were decontaminated by autoclaving prior to sanitizing. Cage boards were changed three times a week at the time when fresh food and water were given (Monday, Wednesday and Friday). Clean and sanitized water bottles and food jars were provided weekly. Suspended caging and banks were sanitized bi-weekly. The animals were observed daily by a staff member and weekly by the attending veterinarian for any signs of health problems. The animals were housed in an AAALAC-accredited facility. Room temperature was maintained at 72° F. (+6° F.) with 10-15 air changes per hour and a 12-hour light cycle.

On day 15 (pup age), the animals received an oral inoculation of streptomycin-resistant S. sobrinus 6715 (ATCC strain #27352) culture. This involved flooding the mouth with 0.2 ml of culture/animal. On day 18 (pup age) the animals were inoculated with S. sobrinus for three consecutive days (age 18, 19 and 20 days). This involved placing 0.1 ml of the S. sobrinus culture on the occlusal surfaces of each of the mandibular molar quadrants, putting 10 cc of this concentration-adjusted culture into each sanitized and filled water bottle, and lightly spraying the bedding with no more than 10 cc of the remaining culture. All water bottles were removed and sanitized 24 hours after inoculum has been added. The inoculums were administered to the animals with a 200 micropipette.

The treatment phase began at day 22 of pup age. Each treatment had a labeled plastic beaker that was designated for that treatment only. Fresh materials (i.e., obtained from the stock supply) were used for each treatment. The dentifrices were mixed in a 1:1 ratio (by weight) with deionized water. Specifically, 10 grams of dentifrice was weighed into a 30 ml beaker; 10 grams of deionized water was then added to the dentifrice. The mixture was then stirred by hand (30 seconds) with a clean micro spatula for the purpose of creating a smooth mixture. The beaker containing the slurry and a small magnetic stirring bar was placed on a magnetic stirrer, which was set at the lowest speed and allowed to stir for four (4) minutes prior to treatm4ent. The slurry was prepared immediately prior to each treatment.

A cotton-tipped applicator was dipped into the slurry (for 2 seconds) and was applied to one-half of the rat's mouth in such a way that the sides of the applicator came into contact with both the mandibular and maxillary molars on one side of the mouth. The treatment was accomplished by using a rolling motion of the sides of the applicator over the mandibular and maxillary molar teeth for 15 seconds. The applicator was dipped into the slurry for the second time (again, for 2 seconds) and the other side of the rat's mouth similarly treated for 15 seconds. A new applicator was used for each animal.

Treatments were administered twice daily for five days with a single daily treatment on weekends. The first treatment each day began at approximately the same time every day, and the second treatment did begin no earlier than six hours after the first treatment. Singular treatments were given at a 24-hour interval on weekends. Treatment materials were stored at room temperature. All treatment products were returned to sponsor at study completion.

One week after the initiation of the inoculation regimen and at study termination, an oral swabbing was taken from each rat using a sterile cotton swab (six-inch, single-tipped applicator). The microorganisms on the mandibular and maxillary molar teeth were sampled, using a rolling motion of the swab for 15 seconds on one side of the mouth, rolled over the tongue, and rolled over the molar teeth on the other side of the mouth for an additional 15 seconds. Immediately after the applicator was removed from the animal's mouth, it was streaked across half of a 100 mm petri plate containing Mitis Salivarius agar to which 200 units/ml of streptomycin sulfate was added. The plates were incubated for 48 hours at 37° C. with 10% CO₂. The colony count taken after the 48 hours of incubation was recorded in the logbook.

The experimental duration of the rat caries studies is three weeks. Immediately prior to termination, all animals were observed for any visual signs of ill health or pathology, individually weighed and an oral swabbing taken to confirm S. sobrinus implantation. The animals were cuthanized by carbon dioxide inhalation. Code numbers were assigned to each animal and the heads were then removed, placed in individual jars along with the code number, and cooked under pressure (10 PSI for 12 minutes). The hemijaws were then removed and freed of all soft tissue.

The cleaned hemijaws (four quadrants) were put into plastic vials with the code numbers taped to the vial. A murexide solution (0.3 g murexide; 300 ml DI H20 and 700 ml of ethanol) was added to each vial and the jaws were allowed to stain overnight. The jaws were then rinsed and allowed to air dry.

The hemijaws were microscopically examined for smooth surface caries, sectioned, and then microscopically examined for sulcal and interproximal caries using the Keyes Method. The scoring method is detailed in Navia, JN, Animal Models in Dental Research, pp 287-290, 1977; and Keyes, PH, J. Dent. Res. 37:1088-1099, 1958. All analyses were performed using SAS statistical software, version 9.4. The groups were compared using analysis of variance (ANOVA), with a fixed effect for group and a random effect for litter. The litter effect was included in the models to reduce a known factor affecting the variability of the measurements. Pair-wise comparisons between groups were made using Tukey's multiple comparisons procedure to control the overall significance level (a=0.05) of the comparisons.

The specific types of data, which were tabulated, and statistically analyzed may include:
1) Mortality Data Experimental Phase
   a. Initial number of animals
   b. Final number of animals
   c. Percent mortality
2) Growth Data Experimental Phase
   a. Initial body weight (mean ±S.E.M.)
   b. Final body weight (mean ±S.E.M.)
3) Caries Experience
   a. Enamel and dentinal involvement of smooth surface (buccal, lingual) lesions (mean ±S.E.M.)
   b. Enamel and dentinal involvement of interproximal lesions (mean ±S.E.M.)
   c. Enamel and dentinal involvement of total smooth surface (buccal, lingual & interproximal) lesions (mean ±S.E.M.)
   d. Enamel and dentinal involvement of sulcal lesions (mean ±S.E.M.)
   e. Total caries involvement combining the scores from the Keyes method of scoring smooth surface, interproximal, and sulcal caries (mean ±S.E.M.)

### Preparation of Oral Care Compositions

The oral care compositions of TABLE 1A were prepared by combining one or more humectants, water, sweetener(s), tin ion source, sodium gluconate, and/or flavor(s) to create a liquid mixture. The liquid mixture was homogenized at 25° C. for 2 minutes. Next, sodium hydroxide (50% solution) was added to the liquid mixture and the liquid mixture was homogenized at 25° C. for 2 minutes. A separate powder mixture was prepared by combining a portion of the calcium ion source and any thickening agents, such as xanthan gum and/or sodium carboxymethylcellulose. The powder mixture was then combined with the liquid mixture. Next, the surfactant, such as sodium lauryl sulfate, was added to the mixture. The contents were homogenized at 25° C. for 2 minutes. The hops extract was then combined with the mixture and homogenized at 25° C. for 2 minutes. Finally, the remaining portion of the calcium ion source and the buffering agent were combined with the mixture and homogenized at 25° C. for 2 minutes.

Preparation of Commercial Oral Care Compositions with Hops Beta Acid

The commercial oral care compositions were combined with hops beta acid by weighing out a portion of commercial oral care composition and mixing in the appropriate amount of hops beta extract. The combined oral care composition was homogenized at 25° C. for at least 2 minutes.

Ex. 1a, 1b, 1c, and 1d, as shown in TABLE 1A, were prepared in accordance with the Experimental Methods, described above. The Hops Beta Acids were supplied by Hopsteiner® as an extract from Humulus lupulus. The Hopsteiner® extract was approximately 45%, by weight of the extract, of hops beta acids and less than 1%, by weight of the extract, of hops alpha acids. Ex. 1a and 1c have 13% water while Ex. 1b and 1d do not have any separately added water. Ex. 1a and Ex. 1b are free from fluoride while Ex. 1c and 1d have sodium monofluorophosphate.

TABLE 1A
Compositions

| | Ex. 1a | Ex. 1b | Ex. 1c | Ex. 1d |
|---|---|---|---|---|
| | (wt %) | (wt %) | (wt %) | (wt %) |
| Sorbitol | 37.98 | — | 36.83 | — |
| Glycerin | — | 49.00 | — | 47.70 |
| Water | 13.00 | — | 13.00 | — |
| Sodium Gluconate | 1.00 | 1.00 | 1.00 | 1.00 |
| Stannous Chloride | 1.10 | 1.10 | 1.10 | 1.10 |
| Calcium Carbonate | 32.00 | 32.00 | 32.00 | 32.00 |
| Xanthan Gum | 0.30 | 0.50 | 0.30 | 0.50 |
| Carboxymethyl | 1.00 | — | 1.00 | — |
| Cellulose | | | | |
| Carbomer | — | 1.00 | — | 1.00 |
| Sodium Lauryl | 1.29 | 1.40 | 1.29 | 1.40 |
| Sulfate | | | | |
| Flavor | 1.00 | 1.10 | 1.00 | 1.10 |
| Sodium Saccharin | 0.50 | 0.40 | 0.50 | 0.40 |
| Stevia Glycosides | — | 0.30 | — | 0.30 |
| Sodium Hydroxide | 0.33 | 1.20 | 0.33 | 1.35 |
| Hops Beta Acid | 0.50 | 0.50 | 0.50 | 0.50 |
| Extract* | | | | |
| Sodium Bicarbonate | 10.00 | 10.00 | 10.00 | 10.00 |
| Titanium Dioxide | — | 0.50 | — | 0.50 |
| Sodium | — | — | 1.15 | 1.15 |
| Monofluorophosphate | | | | |

*Hops Beta Acid Extract supplied by Hopsteiner ®, with 45% hops beta acids and less than 1% hops alpha acids

TABLE 1B
Hops Beta Acids Extract Specification

| Ingredient | Amount (wt %) |
|---|---|
| Hops Beta Acids | 45 ± 2 |
| Hops Alpha Acids | 0.4 ± 0.3 |
| Hops oils | 1.5 ± 0.5 |
| Propylene Glycol | 20 ± 15 |
| Water | <8% |
| pH | 11 ± 0.5 |

TABLE 1B describes the hops beta acid extract provided by Hopsteiner®. Since the hops beta acids are provided as an extract, there can be some variability in the amounts of certain ingredients. However, the extract comprises approximately 45%, by weight of the extract, of the hops beta acids and approximately 0.4%, by weight of the extract, of hops alpha acids. This is dramatically different to previous hops extracts which typically have more hops alpha acids than hops beta acids. Other minor ingredients may be present in the Hops Beta Acid extract.

TABLE 2
Performance of Compositions

| | | % | % | | | | |
|---|---|---|---|---|---|---|---|
| | Total | Reduction | Reduction | | | iPUM- | |
| Treatment Leg | Caries | v Placebo | Predicted | ADA | iPGRM | Ca | HAP |
| Silica Placebo | 42.36 | | | 0 | 0 | 1 | 0 |
| Silica 1000 ppm | 34.08* | 20 | 20 | 1000 | 0 | 1 | 0 |
| F as MFP | | | | | | | |
| Silica 1100 ppm | 27.39* | 35 | 39 | 1100 | 0 | 1 | 0 |
| F as NaF | | | | | | | |
| Ex. 1b | 27.78* | 34 | 38 | 0 | 57 | 3.6 | 33 |
| Ex. 1d | 21.43* | 50 | 65 | 1100 | 57 | 3.9 | 33 |

*Significantly different from Silica Placebo, p < 0.05 as determined by Tukey's adjustment for multiple comparisons

TABLE 3
Fluoride Vector

| | % Reduction | % Reduction | | Performance |
|---|---|---|---|---|
| Treatment Leg | v Placebo | Predicted | ADA | Threshold |
| 2800 ppm F as NaF | 62 | 63 | 2800 | ADA > 650 ppm F as |
| Dentifrice | | | | NaF in a dentifrice |
| 1450 ppm F as NaF | 49 | 45 | 1450 | results in a 29% |
| Dentifrice | | | | reduction in rat caries |
| 1100 ppm F as NaF | 47 | 39 | 1100 | following meta- |
| Dentifrice | | | | analysis. |
| 650 ppm F as NaF | | 29 | 650 | |
| Dentifrice | | | | |
| 500 ppm F as NaF | 24 | 25 | 500 | |
| Dentifrice | | | | |
| 250 ppm F as NaF | 14 | 17 | 250 | |
| Dentifrice | | | | |
| Placebo Dentifrice | — | — | 0 | |

TABLE 4
Antibacterial Agent Vector

| | % Reduction | % Reduction | |
|---|---|---|---|
| Treatment Leg | v Water | Predicted | iPGRM |
| 0.5% Chlorhexidine | 7.5 | 8 | 75 |
| Solution | | | |
| 0.2% Chlorhexidine | 3.8 | 4.2 | 49 |
| Solution | | | |
| Water | — | — | |
| 1.0% Chlorhexidine | 27 | 11.1 | 96 |
| Solution | | | |
| 0.1% Chlorhexidine | 2.4 | 1.3 | 29 |
| Solution | | | |
| Water | — | — | |

TABLE 5
Calcium Vector

| | % Reduction | % Reduction | iPUM- |
|---|---|---|---|
| Treatment Leg | v Water | Predicted | Ca |
| 1.75% CaCl₂ | 5.6 | 10 | 4.2 |
| Composition | | | |
| 2.5% Ca | 1.2 | 5.4 | 3.1 |
| Glycerophosphate | | | |
| Composition | | | |
| Water | — | — | |

TABLE 6
HAP Dissolution Reduction Vector

| | % Reduction | % Reduction | |
|---|---|---|---|
| Treatment Leg | v Control | Predicted | HAP |
| 1000 ppm F as SnF₂ | 3.4 | 9.0 | 13 |
| Dentifrice + 9000 ppm | | | |
| Sn | | | |
| 1000 ppm F as SnF₂ | — | — | 0 |
| Dentifrice | | | |
| 1100 ppm F as SnF₂ | 19 | 10 | 14 |
| Dentifrice + 10,000 ppm | | | |
| Sn | | | |
| 1100 ppm F as SnF₂ | 14 | 10 | 14 |
| Dentifrice + 10,000 ppm | | | |
| Sn | | | |
| 1100 ppm F as SnF₂ | 12 | 12 | 17 |
| Dentifrice + 12,500 ppm | | | |
| Sn | | | |
| 1100 ppm F as NaF | — | — | 0 |
| Dentifrice | | | |

TABLE 7
Arginine Bicarbonate/Calcium Carbonate Example

| | % | % | | | | | |
|---|---|---|---|---|---|---|---|
| | Reduction | Reduction | | | | iPUM- | |
| Treatment Leg | v Control | Predicted | Sig | ADA | iPGRM | Ca | HAP |
| 1100 ppm F as | 22 | 39 | s | 1100 | 0 | 1 | 0 |
| NaF/Silica | | | | | | | |
| Silica F-Placebo | — | — | — | | | | |
| Toothpaste | | | | | | | |
| Arginine Bicarb/Calcium | −8 | 6.3 | ns | 0 | 9 | 3 | 0 |
| Carb | | | | | | | |

The rat caries results, presented in FIG. 1/TABLE 1, indicated that Ex 1b reduced caries relative to sodium fluoride/silica and monofluorophosphate/silica compositions in a placebo-controlled study. Ex. 1b which did not have fluoride, had a total caries reduction equivalent to the sodium fluoride/silica control product; however, it had an unexpectedly large reduction in sulcal caries relative to control. The disclosed compositions were designed to work on the tooth and in the biofilm with a collection of subtherapeutic compositions. This has led to an unexpected reduction in sulcal and interproximal caries with respect to the control compositions. When fluoride is added to the disclosed compositions, as in Ex. 1d, the reduction in caries is enhanced with a notable reduction in smooth surface caries.

It was unexpectedly found that the non-fluoride mechanisms can have a large contribution to the reduction in caries considering that such an approach had not be disclosed in the art. In order to better understand this result, a set of laboratory tests were performed to measure the contribution of each subtherapeutic composition to the composition tested in the rat. Individual methods have been developed to measure the efficacy of compositions along the above described intervention vectors. These methods are the: i) Sn-Free in vitro plaque glycolysis and regrowth method (Sn-Free iPGRM); ii) in vitro plaque uptake method for calcium (iPUM-Ca); iii) F-Free hydroxyapatite solubility reduction method (F-Free HAP); and iv) ADA one-minute fluoride release (ADA). A person of ordinary skill in the art would recognize that some ingredients, such as Sn, have both an antibacterial effect and a HAP surface stabilization effect. Such behavior complicates the analysis of a composition's performance; therefore, the entire contribution of such ingredients is considered through a single mechanism only. Thus, for the purposes of this invention, a composition's antibacterial efficacy should be determined with respect to its Sn placebo. Similarly, for the purposes of this invention, a composition's ability to reduce hydroxyapatite solubility should be determined using the fluoride-free version of the composition (if it contains fluoride). TABLE 2 illustrated the results of the characterization of the novel compositions and control toothpastes using the four different methods indicated above with their corresponding rat caries scores, as disclosed in the Example section.

During the discovery of fluoride, several rat caries tests were run exploring both the effectiveness of fluoride as well as alternatives. The catalogue of rat caries experiments were analyzed to find examples where the use of a single anticaries agent contributed an anticaries effect.

TABLE 3 shows the variation in anticaries efficacy with respect to soluble fluoride content as determined by the ADA method. Using this example and the meta-analysis, a 650 ppm fluoride as sodium fluoride was estimated to effect a reduction in caries of about 29%, or about 25%, or about 30%, with respect to the placebo or water control in rat caries experiments. The table indicates the expected caries reduction in the rat model for the transition from subtherapeutic to therapeutic levels of fluoride delivered as sodium fluoride.

TABLE 4 shows the variation in anticaries efficacy with respect to Sn-Free antibacterial activity as measured by the Sn-Free iPGRM. Since the iPGRM measures reduction in antibacterial efficacy through changes in the pH, non-antibacterial agents that modify pH, such as sodium bicarbonate, can be corrected for when determining the efficacy of the antibacterial agent. This can be done using placebo controls as necessary. The contribution of Sn was measured through the solubility reduction and is excluded from this test. The iPGRM dose response of chlorhexidine is shown in FIG. 2.

TABLE 5 shows the variation in anticaries efficacy with respect to calcium co-ion effect as measured by the iPUM. This method measures the total calcium uptake of both soluble and insoluble sources. If an insoluble source was used, calcium sources were preferred that were substantially more soluble than hydroxyapatite so that they will dissolve preferentially when exposed to plaque acids. For example, dicalcium phosphate or calcium carbonate dissolve readily on exposure to acid while calcium pyrophosphate is a poor source of calcium for the purposes here. Calcium pyrophosphate does not readily dissolve on exposure to acid and is a poor source of calcium for the purposes of protecting teeth from plaque acids through a co-ion effect. Nevertheless, insoluble sources have the advantage of increased residence time in the plaque and can, therefore, provide a bloom of calcium to correspond temporally to the generation of plaque acid. The iPUM dose response of calcium-containing compositions is shown in FIG. 3.

TABLE 6 shows the variation in anticaries efficacy with respect to the F-Free reduction in HAP solubility as measured by F-free HAP. The contribution of the fluoride co-ion effect is measured through the ADA method and is excluded from this test. An example of the F-free HAP response for different levels of Sn in Sn/silica toothpastes is given in FIG. 4.

Finally, the threshold can be defined for which combinations of subtherapeutic compositions result in a therapeutic composition. On average, this transition can be defined for those compositions providing a reduction in rat caries of approximately 29% with a statistically significant reduction in caries with respect to the water or F-placebo/silica toothpaste negative control. Of course, performance can be substantiated using an actual rat caries performance test.

With these methods, the efficacy can be quantified of various compositions along the important anticavity intervention methods. The same analysis can be retrospectively applied to a long history of rat caries experiments conducted during the original development of fluoride-containing anticavity compositions. Approximately 170 rat caries experiments were analyzed containing over 800 individual treatments for the reductions of caries in two different, but similar, rat caries models were analyzed. These experiments were conducted between 1959 and 2019. Rat caries is the preferred animal model for human caries and is included in the US Anticaries Monograph (21 CFR part 355) to ensure the efficacy of fluoride-containing products. It is additionally sensitive to non-fluoride anticaries mechanisms and compositions. Finally, animal models for caries are generally considered interchangeable as long as they have been properly developed.

This retrospective analysis can help define the scope of the present invention. As described herein, the present invention is directed to a combination of subtherapeutic compositions can result in therapeutic composition when tested in total. The examples can allow for the setting of performance thresholds for the subtherapeutic to therapeutic transition in each mechanism as defined by the specified performance test. However, those results, alone, might not anticipate the combined performance of the compositions in a rat caries experiment. Thus, the thresholds for the transition from a subtherapeutic to a therapeutic dose for each mechanism were modeled with historical rat caries data. The analysis gave a model from which can predict reductions in rat caries for combinations of subtherapeutic compositions as described herein. In combination, the examples, performance thresholds, and mathematical model can help define the scope of the present invention.

The retrospective analysis of rat caries experiments was possible because of detailed descriptions of the treatments/ingredients with additional measurements of F content, Sn content, and pH frequently documented in the experimental record. Efficacy measures in the various anticaries mechanism methods described above were either measured directly when the ingredients could be obtained or estimated using the detailed description of the treatments and comparisons to similar present-day compositions. Estimations of rat caries efficacy using the four methods named above resulted in a model correlation coefficient, r², of ˜0.76. The correlation coefficient suggests that 76% of the variation in rat caries efficacy is captured by these methods. The remaining 24% of variation is typically ascribed to biological variation typical in biological methods. Suffice it to say, we believe this represents a good model for the performance of various compositions in rat caries.

The prediction formula is reproduced below for the % reduction in rat caries with respect to water or a silica-based-abrasive, placebo-toothpaste negative control.

$$\% Reduction = 1.77 * sqrt ( ADA / 2 ) + 0.146 * iPGRM + 3.97 * iPUM - Ca + 0.689 * HAP - 6.84 \qquad Formula 1$$

The predicted versus actual values for the retrospective analysis of rat caries data are given in FIG. 5a with a plot of the residuals in FIG. 5b. Additionally, 16 is subtracted from the % Reduction calculated using Formula 1 if a MFP fluoride source is used. This was done because MFP is less effective in the rat caries model.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition comprising:
(a) a first subtherapeutic anticaries agent, wherein the first subtherapeutic anticaries agent comprises metal ion source, wherein the metal ion source comprises stannous chloride; and
(b) a second subtherapeutic anticaries agent, wherein the second subtherapeutic anticaries agent comprises hops beta acid;
wherein the oral care composition is free of fluoride and the first and second subtherapeutic anticaries agents collectively have a therapeutic anticavity benefit.

2. The oral care composition of claim 1, wherein the oral care composition comprises amino acid.

3. The oral care composition of claim 2, wherein the amino acid comprises glycine, alanine, valine, isoleucine, tryptophan, phenylalanine, proline, methionine, leucine, serine, threonine, tyrosine, asparagine, glutamine, cysteine, citrulline, aspartic acid, glutamic acid, lysine, arginine, histidine, or combinations thereof.

4. The oral care composition of claim 1, wherein the therapeutic anticavity benefit is better than the anticavity benefit provided by a control composition, the control composition comprising at least 650 ppm of free fluoride ions.

5. The oral care composition of claim 1, wherein the therapeutic anticavity benefit comprises a rat caries score equal to or greater than the rat caries score of a control composition comprising at least 650 ppm of free fluoride ions.

6. The oral care composition of claim 1, wherein the therapeutic anticavity benefit comprises a reduction in greater than 25% or at least 29% of cavities in a rat caries test.

7. An anticavity, fluoride-free oral care composition, wherein the composition has a rat caries score of 60% or more of a rat caries score of a positive control oral care composition, the positive control oral care composition comprising 1100 ppm of sodium fluoride, wherein the composition comprises stannous chloride and hops beta acid.

8. The anticavity, fluoride-free oral care composition of claim 7, wherein the oral care composition comprises amino acid, metal ion source, or combinations thereof.

9. The anticavity, fluoride-free oral care composition of claim 7, the composition comprising at least two of:
(a) first antibacterial agent;
(b) second antibacterial agent; and/or
(c) calcium.

10. The anticavity, fluoride-free oral care composition of claim 7, the composition comprising:
(a) antibacterial agent, wherein the antibacterial agent comprises hops beta acid;
(b) buffering agent; and
(c) calcium.

11. The anticavity, fluoride-free oral care composition of claim 10, wherein the oral care composition comprising amino acid.

12. The anticavity, fluoride-free oral care composition of claim 11, wherein the amino acid comprises glycine, alanine, valine, isoleucine, tryptophan, phenylalanine, proline, methionine, leucine, serine, threonine, tyrosine, asparagine, glutamine, cysteine, citrulline, aspartic acid, glutamic acid, lysine, arginine, histidine, or combinations thereof.

13. The anticavity, fluoride-free oral care composition of claim 10, wherein the calcium comprises calcium carbonate, calcium bicarbonate, calcium phosphate, calcium chloride, or combinations thereof.
